(19) United States
(12) Patent Application Publication
(10) Pub. No.: US 2024/0309326 A1
(43) Pub. Date: Sep. 19, 2024

(54) Method of Isolating Adipose-Derived Cell Lines and Uses Thereof

(71) Applicant: AGENCY FOR SCIENCE, TECHNOLOGY AND RESEARCH, Singapore (SG)

(72) Inventors: Jian Ming Lamony Chew, Singapore (SG); Khin Oo Angela Lwin, Singapore (SG); Yeh Qi Cheryl Wong, Singapore (SG); Shigeki Sugii, Singapore (SG)

(21) Appl. No.: 18/577,873

(22) PCT Filed: Jul. 8, 2022
(86) PCT No.: PCT/SG2022/050482

§ 371 Date: Jan. 9, 2024

(30) Foreign Application Priority Data
Jul. 9, 2021 (SG) 10202107584T

(51) Int. Cl. C12N 5/077

(52) U.S. Cl. CPC C12N 5/0653; C12N 2500/36; C12N 2500/38; C12N 2501/33; C12N 2501/39; C12N 2531/00

(57) ABSTRACT

This technology relates to a method of isolating adipose-derived cell lines from an animal, a method of isolating adipocytes from an animal, and isolated adipose-derived cell lines thereof.

### FIELD OF THE INVENTION

The present invention relates generally to the field of cell biology. In particular, the present invention relates to methods of isolating adipose-derived cell lines and culturing these cell lines for further use.

### BACKGROUND

The increasing world population places a heavy burden on the food demand. To feed a population of nearly 10 billion by 2050, the current food supply system from over 570 million farms imposes intense pressure on the terrestrial and aquatic environment. In particular, the environmental impacts of animal products far exceed those of vegetables, contributing over 80% of the world's farmland and over 56% of greenhouse gas emissions from food production.

Cellular agriculture is an emerging field that aims to manufacture agricultural products derived from cell culture technology rather than traditional farming and harvesting of livestock or plants. Cellular agriculture has promising potential in large scale production of food products, with lesser environmental burden. In addition, cellular agriculture offers benefits such as no slaughtering of animals, easier pathogen control, and potentially antibiotic-free and pollution-free production in the production process.

Cell-based meat, also referred to as cultured meat, clean meat, lab-grown meat or cultivated meat, utilizes cellular agriculture techniques and biomanufacturing technology of animal cell lines in order to create edible food structures similar to animal meats. While muscles being the predominant constituent of meat products, fat, especially intramuscular fat, contributes to juiciness and tenderness in meat, hence improving palatability and satiation. According to United States Department of Agriculture, fat also serves as an efficient energy source that provides 8.8 kcal/g of energy in food, compared to 4.1 kcal/g for carbohydrate and protein.

Rapid advancement in research and marketing efforts, as well as public attention has been focused strongly on cell-based meat development on the production of various proteins. However, little is done to produce cell-based fat that can be used to improve the taste, nutritional value, and texture of cell-based meat product.

### SUMMARY OF INVENTION

In one aspect, the present disclosure refers to a method of isolating adipose-derived cell lines from an animal, comprising: (a) obtaining an adipose tissue sample from an animal; (b) collecting stromal vascular cells from the adipose tissue sample of (a); (c) expanding the stromal vascular cells of (b) in the presence of a serum; (d) conducting clonal selection of the expanded stromal vascular cells based on the growth of the cells; and (e) isolating one or more adipose-derived cell lines based on the selection result in (d).

In another aspect, the present disclosure refers to an adipogenesis induction composition comprising: a linoleic acid-oleic acid albumin (LAOA), an insulin, a serum, and optionally at least one, at least two, or all of 3-isobutyl-1-methylxanthine (IBMX), dexamethasone, and indomethacin.

In another aspect, the present disclosure refers to a method of obtaining adipocytes from an animal, comprising: (a) isolating adipose-derived cells from a tissue sample of the animal; (b) culturing the adipose-derived cells in the presence of the adipogenesis induction composition as disclosed herein; and (c) obtaining adipocytes from the cell culture of (b).

In another aspect, the present disclosure refers to an adipose-derived cell line obtained or obtainable by the method as disclosed herein, wherein the cell line is characterized by not entering senescence state after at least 16 passages.

In another aspect, the present disclosure refers to a kit for adipogenesis induction of adipose-derived animal cells in vitro, comprising: (a) the composition as disclosed herein; and (b) the adipose-derived cell line as disclosed herein or an isolated adipose-derived cell line obtained according to the method as disclosed herein.

In another aspect, the present disclosure refers to an adipose cell produced by the method as disclosed herein.

In another aspect, the present disclosure refers to a food product comprising the cell line as disclosed herein, or the adipose cell as disclosed herein.

In another aspect, the present disclosure refers to a lipid composition obtained from the cell line as disclosed herein, or a fat isolated from the adipose cell as disclosed herein.

In another aspect, the present disclosure refers to the use of the lipid composition as disclosed herein in cosmetics, food additives, or nutritional supplements.

### BRIEF DESCRIPTION OF THE DRAWINGS

The invention will be better understood with reference to the detailed description when considered in conjunction with the non-limiting examples and the accompanying drawings, in which:

FIG. 1A provides an overview of an exemplary procedure of the cultured meat production. Tissues obtained directly from animals are processed, from which stable cell lines are developed. The cell lines are subsequently cultured in large scale to expand in quantity, for example, with microcarrier to support adherence of cells. Large quantity of cells after expansion are differentiated into desired cell types, for example, muscle cells and adipocytes, and combined with edible scaffolds to produce cultured meat. FIG. 1B provides an exemplary workflow for producing cell-based meat from isolated fish cells, showing cell line development as the first step of cultivated meat for production of adipocytes for cultivated meat. FIG. 1C provides a table for comparison between a fat cell and a fat molecule based on their biological, physical, and chemical properties and nutritional facts.

FIG. 2 provides microscopic images showing isolated adipose-derived stem cells developed from animal tissue samples. FIG. 2A shows the growth of adipose tissues isolated from Pangasianodon hypophthalmus in cell culture using the method disclosed herein. The presence of fish serum contributes to the survival of freshly isolated adipose-derived cells and thus is required in the isolation of adipose-derived cells. FIG. 2B provides images of successfully isolated and grown adipose-derived cells according to the methods disclosed herein from different animals, for example, four species of fishes using the same procedures as disclosed herein. Scale bar represents 200 μm.

FIG. 3 provides exemplary images showing the morphology of adipose-derived cells before and after clonal selection based on the method disclosed herein. The cells remain consistently in its spindle-shape before and after clonal selection. Left panel shows cells at 4× magnification (scale bar represents 200 μm) and the right panel shows cells at 10× magnification (scale bar represents 80 μm).

FIG. 4 shows proliferation rate of adipose-derived cells in the presence of fish serum of various concentrations. The isolated adipose-derived cells obtained based on the method disclosed herein need to be cultured in the presence of a serum. To determine the suitable amount of serum used to support the growth and expansion of cells, Pangasianodon hypophthalmus cell lines were grown in complete media containing 20% FBS and titrating concentration of fish serum. Cell proliferation of the cell lines were measured using CellTiter-Blue Assay over 5 days. Data are expressed as a percentage of the maximum Fluorescence Intensity for each cell line and shown as mean±SEM of 4 cell lines. Asterisk (*) indicates p<0.05 as compared to cell lines grown in 0% fish serum using one-way ANOVA, followed by Tukey post-hoc test.

FIG. 5 describes the proliferation rate of adipose-derived cells in the presence of 15% FBS and 0.1% serum of various animals. The isolated adipose-derived cells obtained based on the method disclosed herein were cultured in the presence of Pangasius or Tilapia fish serum. To determine if fish serum is advantageous to maintain the proliferation of fish cell line, FIG. 5 shows the growth rate of the exemplary cell line Ph9F-1x in complete growth media containing 15% FBS and 0.1% of different animal sera over 4 days. Data are shown as mean±SEM of 3 independent wells. Asterisk (*) indicates p<0.05 using one-way ANOVA, followed by Tukey post-hoc test.

FIG. 6 describes the proliferation rate of adipose-derived cells in the presence of FBS of various concentrations. The isolated adipose-derived cells obtained based on the method disclosed herein need to be cultured in the presence of FBS. To determine the suitable amount of FBS, FIG. 6A shows Pangasianodon hypophthalmus adipose-derived cell lines were grown in complete media containing 0.1% fish serum and titrating concentration of FBS over 5 days. FIG. 6B shows Anguilla japonica adipose-derived cell lines grown in complete media containing titrating concentration of FBS over 4 days. Cell proliferation of the cell lines were measured using CellTiter-Blue Assay. Data is expressed as a percentage of the maximum FI for each cell line and shown as mean±SEM of 4 cell lines. Asterisk (*) indicates p<0.05 using one-way ANOVA, followed by Tukey post-hoc test.

FIG. 7 describes the proliferation of several adipose tissue-derived cell lines obtained over time. Exemplary Pangasianodon hypophthalmus cell lines were grown in complete media containing 0.1% fish serum and 15% FBS over 5 days. Another different species Anguilla japonica cell lines were grown in complete media containing 10% FBS over 4 days. Proliferation of the cell lines were measured using CellTiter-Blue Assay. Data are shown as mean±standard deviation of 3 independent wells. The proliferation of the cell lines demonstrates variations, and the cell lines are categorised according to their proliferation rate to fast growing cell lines, and slow growing cell lines, respectively.

FIG. 8 shows the absence of mycoplasma contamination in the isolated adipose-derived cell lines disclosed in the present invention. The presence of mycoplasma in the cell culture supernatant was detected using MycoALERT™ PLUS mycoplasma detection kit. Relative luminescence unit (RLU) of less than 1.0 is regarded as negative. Data are shown as mean of 2-4 replicates.

FIG. 9 provides exemplary base media that are commonly available for culturing the isolated adipose-derived cell lines. Exemplary Pangasianodon hypophthalmus adipose-derived cell line proliferates significantly faster in DMEM and advanced DMEM as compared to Leibovitz's L15, Mesencult ACF Plus and Stempro MSC SFM CTS. The tested cell lines were grown in different media containing 0.1% fish serum. Cell proliferation of the cell lines were measured using CellTiter-Blue Assay over 5 days. Data are shown as mean±SEM of 4 cell lines. Asterisk (*) indicates p<0.05 using one-way ANOVA, followed by Tukey post-hoc test.

FIG. 10 shows the cell count over time for the average population doubling time of exemplary adipose-derived cell lines (Pangasianodon hypophthalmus and Anguilla japonica) isolated according to the methods disclosed herein. Fast growing cell lines Ph9F-1x from Pangasianodon hypophthalmus and Aj1C-1x from Anguilla japonica were grown for 72 hours. The nuclei were stained with CyQUANT direct cell proliferation assay and imaged every 24 hours. The doubling time for the two cell lines are calculated to be about 13.7 and about 31.2 hours, respectively. Data are shown as mean±standard deviation of 3 independent wells.

FIG. 11 shows the morphology and cell count growth over time of a fast-growing fish adipose-derived cell line, Ph9F-1x, for example, at passage 21, 54, and 113 (scale bar represents 200 μm). The isolated adipose-derived cell lines disclosed herein are able to expand after large number of passages and retaining the same morphological characteristics of spindle-shaped appearance. Cells were grown in culture until about 70% confluent before passaging. Cell counts were measured by staining the nuclei with CyQUANT direct cell proliferation assay and imaging every 24 hours for 3 days. Data are shown as mean±standard deviation of 4 independent wells.

FIG. 12 shows adipogenesis induction of an exemplary fast-growing adipose-derived cell line, Ph9F-1x, differentiating into mature adipocytes after treatment with an exemplary induction media of DMEM and adipogenic induction cocktails with 100 μM of linoleic acid-oleic acid albumin (LAOA). FIG. 12A provides images of cell nuclei stained with Hoechst 33342 and neutral lipids stained with AdipoRed. Images are taken at 10× magnification, and the scale bar represents 100 μm. FIG. 12B shows the percentage of adipogenesis (determined by the number of cells expressing neutral lipids) and FIG. 12C shows total lipid accumulation (determined by the total fluorescence intensity of AdipoRed) of treated cells. Data are shown as mean±standard deviation of 3 independent wells. Asterisk (*) indicates p<0.05 using one-way ANOVA, followed by Tukey post-hoc test.

FIG. 13 provides exemplary images of exemplary Anguilla japonica and Scortum barcoo cell lines that differentiate into mature adipocytes after adipogenesis induction. The cell lines are treated with exemplary DMEM containing adipogenic induction cocktails with 100 μM of LAOA. Cell nuclei were stained with Hoechst 33342 and neutral lipids were stained with AdipoRed. Images are taken at 10× magnification, and the scale bar represents 100 μm.

FIG. 14 describes another example of an adipose-derived cell line, Ph9F-1x, differentiating into mature adipocytes. FIG. 14A provides a schematic diagram illustrating the exemplary adipogenesis protocol using a FBS-containing DMEM-based adipogenic induction cocktail and a FBS-free Essential 6-based adipogenic induction cocktail. The exemplary DMEM-based adipogenic induction cocktail used herein contains high glucose DMEM, FBS, fish serum, antibiotics, insulin, dexamethasone, IBMX, indomethacin, and LAOA. The exemplary Essential 6-based adipogenic induction cocktail used herein contains Essential 6, fish serum, dexamethasone, IBMX, indomethacin, and LAOA. FIG. 14B provides exemplary images of adipocytes after induction using the induction media illustrated in FIG. 14A. Cell nuclei were stained with Hoechst 33342 and neutral lipids were stained with AdipoRed. Images are taken at 10× magnification, and the scale bar represents 100 μm. FIG. 14C shows the percentage of adipogenesis calculated by the number of cells expressing neutral lipids and total lipid accumulation (determined by the total fluorescence intensity of AdipoRed). Data are shown as mean±standard deviation of 3 independent wells. Asterisk (*) indicates p<0.05 using Students' T-test.

FIG. 15 shows the yield of adipocytes and level of lipid accumulation in the adipocytes after adipogenic induction of isolated adipose-derived cells. Exemplary cell line Ph9F-1x was treated with one of the disclosed exemplary induction media comprising Essential 6 media, fish serum, adipogenic induction cocktail, and LAOA over 9 days. Cell nuclei were stained with Hoechst 33342 and neutral lipids were stained with AdipoRed on different days over the period of induction. Images are taken at 10× magnification, and the scale bar represents 100 μm. Data are shown as mean±standard deviation of 3 independent wells. Asterisk (*) and hash (#) indicate p<0.05 as compared to Day 6 and Day 9 respectively using one-way ANOVA, followed by Tukey post-hoc test.

FIG. 16 shows various combinations of components used in adipogenic induction. Highest level of lipid accumulation was observed in fish adipose-derived cell lines after treatment with Essential 6, fish serum, LAOA, IBMX, and dexamethasone for 6 days. Data are shown as mean±standard deviation of 3 independent wells. Asterisk (*) indicates p<0.05 as compared to treatment with IBMX, dexamethasone, indomethacin, and LAOA using one-way ANOVA, followed by Tukey post-hoc test.

FIG. 17 provides the percentage of cells undergoing adipogenesis with the treatment of different concentrations of LAOA in an exemplary adipose-derived cell line. The adipogenesis induction cocktail as disclosed herein requires LAOA. To titrate the amount of LAOA suitable for adipogenesis induction, exemplary cell line Ph9F-1x was treated with Essential 6 media, fish serum, IBMX, dexamethasone, and different concentrations of LAOA over 6 days. Cell nuclei were stained with Hoechst 33342 and neutral lipids were stained with AdipoRed. Images are taken at 10× magnification, and the scale bar represents 100 μm. Data are shown as mean±standard deviation of 3 independent wells. Asterisk (*) indicates p<0.05 using one-way ANOVA, followed by Tukey post-hoc test.

FIG. 18 shows supplementation of LAOA at different time points of adipogenesis, for example, from Day 0 to 3, Day 3 to Day 6, or Day 0 to Day 6 of adipogenesis to investigate at which time periods LAOA is necessary for adipogenesis induction. An exemplary cell line Ph9F-1x was treated with 100 μM of LAOA from (A) Day 0 to Day 3, (B) Day 3 to Day 6, or (C) Day 0 to Day 6, in the presence of Essential 6 media, fish serum, IBMX, and dexamethasone over 6 days. Cell nuclei were stained with Hoechst 33342 and neutral lipids were stained with AdipoRed. Images are taken at 10× magnification, and the scale bar represents 100 μm. FIG. 18D and FIG. 18E shows the percentage of adipogenesis determined by the number of cells expressing neutral lipids and total lipid accumulation determined by the total fluorescence intensity of AdipoRed, respectively. Data are shown as mean±standard deviation of 3 independent wells. Asterisk (*) indicates p<0.05 using one-way ANOVA, followed by Tukey post-hoc test.

FIG. 19 shows the adipogenesis percentage in adipose-derived cell lines using LAOA, oleic acid albumin (OAA), or linoleic acid albumin (LAA), respectively. Exemplary cell line Ph9F-1x was treated with 100 μM of LAOA, OAA, or LAA, in the presence of Essential 6 media, fish serum, IBMX, and dexamethasone over 6 days. Cell nuclei were stained with Hoechst 33342 and neutral lipids were stained with AdipoRed. Images are taken at 10× magnification, and the scale bar represents 100 μm. Data are shown as mean±standard deviation of 3 independent wells. Asterisk (*) indicates p<0.05 using one-way ANOVA, followed by Tukey post-hoc test.

FIG. 20 summaries the changes in expression profile of genes involved adipogenesis and lipogenesis on Day 3 and/or Day 6 of adipogenic induction. Exemplary cell line Ph9F-1x was treated with Essential 6 media, fish serum, LAOA, IBMX, and dexamethasone over 6 days. Data are shown as mean±standard deviation of 3 independent wells. Asterisk (*) indicates p<0.05 using one-way ANOVA, followed by Tukey post-hoc test.

FIG. 21 describes the use of serum in the adipogenesis induction cocktail at different time points of adipogenesis. As described in the methods disclosed herein, the adipose-derived cells are cultured in the presence of serum. It is investigated whether serum is required during the entire duration of induction. Exemplary cell line Ph9F-1x from fish was treated (A) without fish serum, with fish serum (B) from Day 0 to Day 3, or (C) from Day 0 to Day 6, in the presence of Essential 6 media, LAOA, IBMX, and dexamethasone over 6 days. Cell nuclei were stained with Hoechst 33342 and neutral lipids were stained with AdipoRed. Images are taken at 10× magnification, and the scale bar represents 100 μm. FIG. 21D provides the percentage of adipogenesis determined by the number of cells expressing neutral lipids and FIG. 21E shows the total lipid accumulation in the treated cells determined by the total fluorescence intensity of AdipoRed. Data are shown as mean±standard deviation of 3 independent wells. Asterisk (*) indicates p<0.05 using one-way ANOVA, followed by Tukey post-hoc test.

FIG. 22 shows the effects of supplementation of different vitamins in the adipogenesis of adipose-derived cell line. Exemplary cell line of Ph9F-1x was treated with 100 μM of α-tocopherol acetate, D-pantothenic acid, vitamin D3, or biotin in the presence of Essential 6 media, fish serum, LAOA, IBMX, and dexamethasone over 6 days. Data are shown as mean±standard deviation of 3 independent wells.

FIG. 23 shows the level of lipid intensity in another exemplary adipose-derived cell line Aj1C-1x at different time points during adipogenic differentiation. The Anguilla japonica cell line was treated with Essential 6 media, adipogenic induction cocktail, and LAOA over 5 days. Neutral lipids were stained with AdipoRed and the fluorescence intensity were measured daily. Data are shown as mean±standard deviation of 3 independent wells. Asterisk (*) and hash (#) indicate p<0.05 using one-way ANOVA, followed by Tukey post-hoc test.

FIG. 24 shows different concentrations of LAOA in adipogenesis induction and their effects in the efficiency of adipogenesis and total lipid intensity in another exemplary cell line, Aj1C-1x from Anguilla japonica. The cell line was treated with Essential 6 media, adipogenic induction cocktail, and LAOA over 3 days. Cell nuclei were stained with Hoechst 33342 and neutral lipids were stained with AdipoRed. Images are taken at 10× magnification, and the scale bar represents 100 μm. Data are shown as mean±standard deviation of 3 independent wells. Asterisk (*) indicates p<0.05 using one-way ANOVA, followed by Tukey post-hoc test.

FIG. 25 describes the effects of supplementation of DHA and/or EPA on the efficiency of adipogenesis of adipose-derived cell line. Exemplary cell line Ph9F-1x was treated with different concentrations of (A) DHA, and (B) EPA, or (C) a combination of 50 μM of DHA and 50 μM of EPA, in the presence of Essential 6 media, fish serum, and LAOA for the last 3 days of adipogenesis. Data are shown as mean±standard deviation of 3 independent wells.

FIG. 26 shows the upregulation of adipogenic genes, for example, PPARγ and C/EBPβ, on Day 3 and Day 6 of adipogenic induction with DHA and EPA containing adipogenic induction cocktail. Exemplary cell line Ph9F-1x was treated with Essential 6 media, fish serum, LAOA, IBMX, dexamethasone, DHA, and EPA over 6 days. Data are shown as mean±standard deviation of 3 independent wells. Asterisk (*) indicates p<0.05 using Students' T test.

FIG. 27 provides images of exemplary cell line Ph9F-1x, which retains adipogenic potential at high passage number of 29 and 104. Ph9F-1x at passage 29 and 104 were treated with Essential 6 media, adipogenic induction cocktail, and LAOA over 6 days. Cell nuclei were stained with Hoechst 33342 and neutral lipids were stained with AdipoRed. Images are taken at 10× magnification, and the scale bar represents 100 μm. Data are shown as mean±standard deviation of 3 independent wells. Asterisk (*) indicates p<0.05 using one-way ANOVA, followed by Tukey post-hoc test.

FIG. 28 provides an example of culturing of isolated cell line Ph9F-1x using Cytodex 1 microcarrier in 3-dimension stirring spinner flasks. The exemplary cell line Ph9F-1x adhered, grew, and differentiated in the microcarrier. FIG. 28A shows the viability and cell counts of Ph9F-1x cells, which were grown on Cytodex 1 for 3 days and treated with TrypLE Express for 30 minutes. Cells were stained with Trypan blue and counted using LUNA automated cell counter. FIG. 28B shows image of cell nuclei adhering to a microcarrier were stained with Hoechst 33342 and imaged with Nikon A1R+si Confocal Microscope. FIG. 28C describes the adipogenesis of the cultured cells treated with Essential 6 media, adipogenic induction cocktail, and LAOA over 6 days in the 3-dimensional culture system. Cell nuclei were stained with Hoechst 33342 and neutral lipids were stained with AdipoRed. Images are taken using N-STORM/TIRF microscope. Scale bar represents 100 μm.

### DEFINITIONS

In general, the term “fat” when used, can be understood as referring to either “fat tissue” or “fat molecule”, depending on the context of discussion. As used herein, the term “fat” refers to “fatty tissue” or “adipose tissue”, which is a connective tissue consisting mainly of fat cells. It is found mainly under the skin (subcutaneous fat) but also in deposits within or between the muscles (intermuscular fat and intramuscular fat), in the internal organs and in their membrane folds (visceral fat, for example, mesenteric fat), and bone marrow. The fat stored in adipose tissue comes from dietary fats or is produced in the animal body.

As used herein, the term “fat cell”, also known as “adipocyte” or “lipocyte”, refers to a specialized cell of adipose tissue that stores excess energy in the form of triglyceride droplets. As used herein, the term “triglyceride” refers to an energy-rich compound made up of a single molecule of glycerol and three molecules of fatty acid. Triglyceride serves as a major component of animal and plant oils and fats. Animal triglycerides are important energy source and present in adipose tissues, bloodstream, and heart muscle.

As used herein, the term “fatty acid” refers to aliphatic monocarboxylic acids derived from or contained in esterified form in an animal or vegetable fat, oil, or wax. Natural fatty acids commonly have a chain of 4-28 carbons (usually unbranched and even-numbered), which may be saturated or unsaturated.

As used herein, the term “oil” refers to a triglyceride that is liquid at room temperature. The main difference between fats and oils is that fats are composed of high amounts of saturated fatty acids which will take a solid form at room temperature whereas oils are composed of mainly unsaturated fatty acids which will take a liquid form at room temperature. An increase in the percentage of shorter-chain fatty acids and/or unsaturated fatty acids lowers the melting point of a fat or oil.

As used herein, the term “stromal vascular fraction” refers to a heterogeneous fraction obtained from adipose tissue, which comprises several types of cells including adipose stromal cells, adipocyte progenitors, fibroblasts, immune cells, epithelial cells, endothelial cells, and other cell types associated with the circulatory and nervous systems. There are various techniques to extract these cells by breaking up the fat tissue and facilitate the harvesting of these cells. The term “stromal vascular cells” refers to the heterogenous cells contained in the stromal vascular fraction collectively.

As used herein, the term “growth rate” for cultured cells refers broadly to the rate of a population of cells to increase in number within a certain time period. As described herein, for example, the growth rate of cells can be quantified by doubling time, which is the time it takes for a population to double in number.

As disclosed herein the term “lipid” refers to a macro biomolecule that is soluble in nonpolar solvents, for example, hydrocarbons. Lipid includes fatty acids, waxes, sterols, fat-soluble vitamins (such as vitamins A, D, E, and K), monoglycerides, diglycerides, triglycerides, and phospholipids.

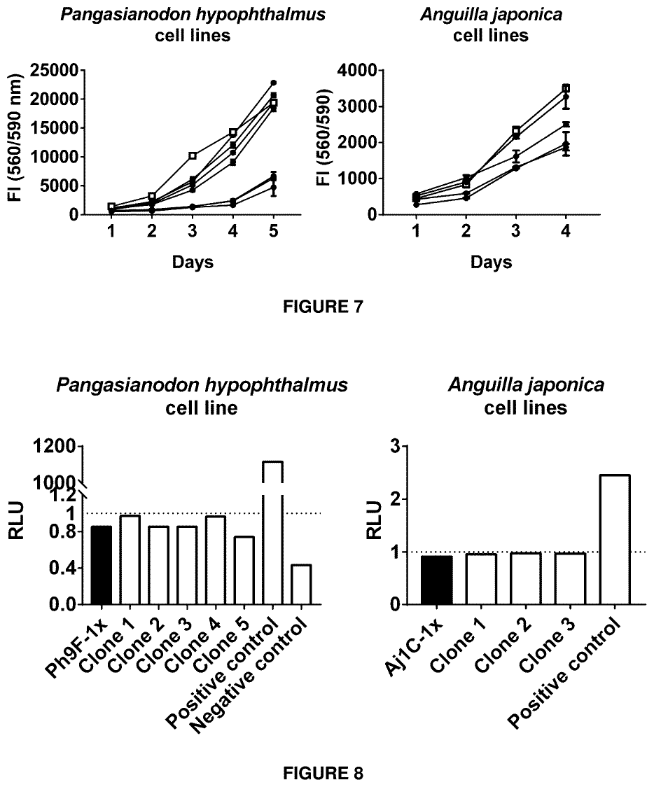

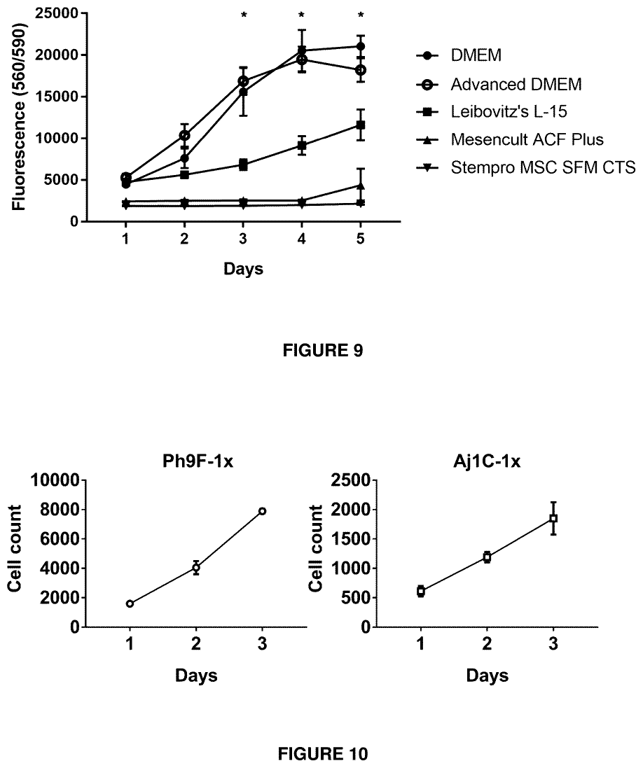

As used herein, the term “fast-growing cell lines” refers to cell lines having growth rates higher than the median growth rates of all isolated cell lines of the same species. As used herein, the term “slow-growing cell lines” refers to cell lines having growth rates lower than the median growth rates of all isolated cell lines of the same species. For example, as shown in FIG. 7 a few cell lines isolated from Pangasianodon hypophthalmus and Anguilla japonica display steep increase in the number of cells over time, while others showing a flatter growth curve.

As used herein, the term “cultivated meat” or “cultured meat” refer to an animal meat (including seafood and organ meats) which is produced by cultivating or culturing animal cells in vitro, without the need to capture or farm animals as a food source for protein and animal fat. A cultivated meat or cultured meat comprises, typically, one or more types of cultured cells (for example, muscle cells) seeded on an edible or biodegradable scaffold, and additives, for example, additional nutrients or flavouring agents.

As used herein, the term “edible scaffold” or “biodegradable scaffold” refers to the three-dimensional mesh used in the manufacture of cultured or cultivated meat products. The “edible scaffold” or “biodegradable scaffold” provides structural support, as well as mechanical and biochemical cues to the cells in vitro. Commonly used scaffold components include, for example, collagen and gelatin.

### DETAILED DESCRIPTION OF THE PRESENT INVENTION

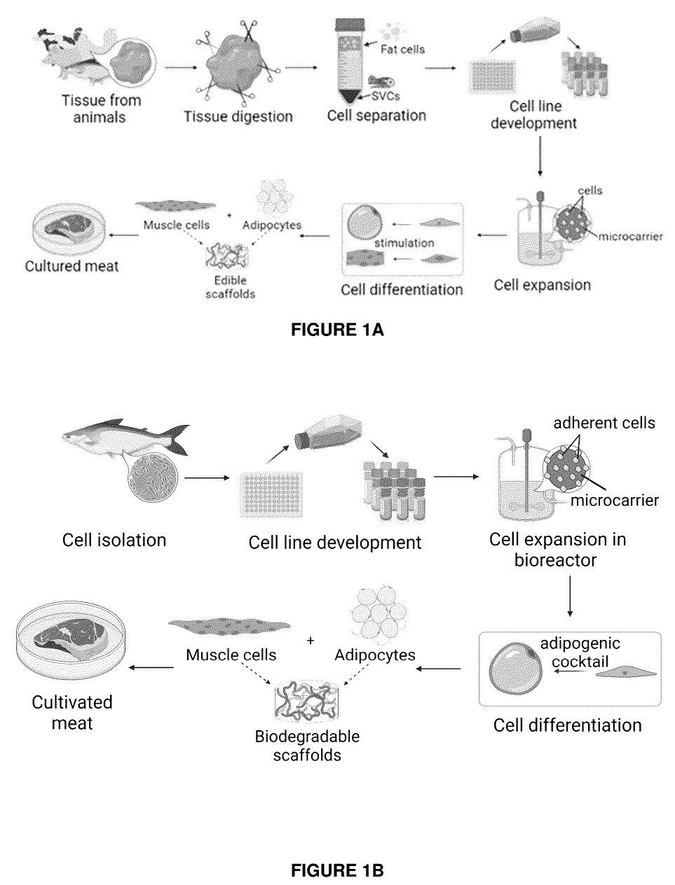

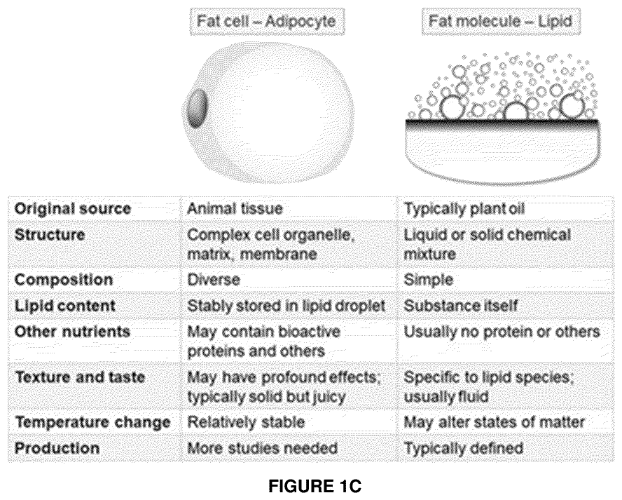

The increasing environmental pressure to feed a growing population calls for solutions from cellular agriculture, which is an emerging field that aims at producing agricultural products, in particular, meats, from cell culture technology. The overall procedure of the cultured meat production is illustrated in FIG. 1A. Typically, cells are isolated from biopsies of edible animal species, and stem/progenitor cell lines are derived that have capability to exponentially grow and differentiate into mature cell types such as myoblasts and adipocytes. After cells are expanded in large-scale culture, stem/progenitor cells are stimulated to become mature cell types, for example, muscle cells. These cells may be then combined with edible scaffolds and other food grade materials to constitute meat for ultimate consumption.

While muscles being the predominant constituent of meat products, fat contributes to palatability and nutritional values in meat. In the nutrient point of view, fat often refers to lipid molecules such as triglycerides, phospholipids, sterols, and free fatty acids. In contrast, when referring to solid components of meat as discussed in the present disclosure, fat typically means fat (adipose) cells or tissue. Differences in characteristics of fat cell (adipocyte) versus fat molecule (lipid) in terms of food ingredients are summarized in FIG. 1B. Adipocyte and its stem/progenitor cell from food related species are predominant sites of lipids in animal meat products.

Despite rapid advancement in research and marketing efforts in the development of cell-based meat targeting proteins, little is done to produce cell-based fat. Stable and fast-growing cell lines are necessary for obtaining sufficient amount of cultured meat fat for large-scale manufacturing. However, so far, there is limited progress in isolating and culturing cell lines derived from animal fat. This disclosure provides methods, isolated cell lines, and products thereof, related to isolated adipose-derived cells.

As disclosed herein, methods of isolating adipose-derived cell lines from an animal are described. In one example, the present disclosure describes methods of isolating adipose-derived cell lines from an animal, comprising obtaining an adipose tissue sample from an animal. The adipose tissue sample obtained herein can be, but is not limited to a fat tissue, a visceral fat, a subcutaneous fat, a bone marrow fat, a mesenteric fat, or an intramuscular/intermuscular fat comprised in a muscle.

In another example, the present disclosure describes methods of isolating adipose-derived cell lines from an animal, comprising obtaining an adipose tissue sample from an animal and collecting stromal vascular cells from the adipose tissue sample obtained. As understood by a person skilled in the art, methods of collecting stromal vascular cells from a tissue sample are know in the art. For example, the stromal vascular fraction comprising heterogeneous types of stromal vascular cells can be isolated from the tissue sample by enzymatic digestion. The adipose tissue sample can be fragmented by cutting or mincing and digested with collagenase type IV, or with collagenase type IV in combination with dispase II. Alternatively, the stromal vascular fraction comprising heterogeneous types of stromal vascular cells can be isolated from the tissue sample by explant culturing. The adipose tissue sample can be fragmented by cutting or mincing and cultured until the stromal vascular cells migrate and proliferate outside the tissue sample.

In another example, the present disclosure describes methods of isolating adipose-derived cell lines from an animal, comprising obtaining an adipose tissue sample from an animal; collecting stromal vascular cells from the adipose tissue sample obtained; and expanding the stromal vascular cells collected in the presence of a serum. The term “serum”, as used herein refers to the fluid and solute component of blood which does not play a role in clotting. It may be defined as blood plasma without the clotting factors (such as fibrinogen and prothrombin), or as blood with all cells and clotting factors removed. A serum can be obtained by methods known in the art, for example, via centrifugation to separate from blood cells. The amount of serum is 0.1%-2%, 0.2%-1.8%, 0.4%-1.6%, 0.6%-1.2%, or 0.8-1%. In some further examples, the serum is a fish serum. The usage of serum from the same host (i.e. chicken serum for chicken cells) are not commonly used for adipocytic cell isolation. Most prior studies used bovine serum only as it is widely available and affordable. It was found by the inventors that autologous serum or serum from an animal of the same phylum (i.e. fish serum, avian serum, porcine serum) play critical role in SVF isolation. In some examples, the stromal vascular cells are expanded in the presence of a serum containing growth medium. In some examples, the stromal vascular cells are expanded in the presence of a serum containing complete growth medium. In further examples, the serum containing complete growth medium comprises a base medium. It is understood by a person skilled in the art that the base medium can be any medium suitable for animal cell culture, including, but not limited to: Eagle's minimal essential medium (MEM), Dulbecco's modified Eagle's medium (DMEM), Iscove basic DMEM (IMDM), Roswell Park Memorial Institute medium (RPMI), 199 medium, 109 medium, Ham's F-10 medium, Ham's F-12 medium, McCoy's 5A Medium, Leibovitz's L-15 medium, advanced DMEM, Mesencult ACF Plus medium and Stempro MSC SFM CTS medium. It is understood by a person skilled in the art that the composition can be used in combination with other commonly used supplements in a cell culture, for example, amino acids, vitamins, inorganic salts, glucose, growth factors, hormones, and attachment factors. It is also understood that the medium disclosed herewith maintains a suitable pH and osmolality for cell growth. For a person skilled in the art, it is easily understood that the temperature, humidity and gaseous atmosphere of cell culture depends on the type of cells, and can be optimized routinely. For example, the cells are cultured at about 20-37° C. for about 1-1.5 hours.

In some examples, the stromal vascular cells are collected by enzymatic digestion. For example, the stromal vascular cells can be isolated by collagenase or collagenase and dispase. In one example, the stromal vascular cells are isolated using 1 mg/mL type I collagenase (Worthington) in Hank's buffered salt solution (HBSS) containing 1% BSA, and 50 mg/mL glucose. In another example, the stromal vascular cells are isolated using 1 mg/ml of collagenase type IV in HBSS containing 2.5 mM calcium chloride. In yet another example, the stromal vascular cells are isolated using 0.5 mg/ml of collagenase type IV and 2.4 mg/ml of dispase II in HBSS containing 2.5 mM calcium chloride. In other examples, the stromal vascular cells are collected by explant culture. For example, the stromal vascular cells can be isolated by culturing the fragmented tissue sample in a growth media and collecting the cells migrating out of the tissue sample. In further examples, the stromal vascular cells are collected by filtering after enzymatic digestion or explant culturing of the tissue sample. In some examples, the stromal vascular cells are filtered by a nylon filter.

In some examples, the collected stromal vascular cells are expanded in the presence of a Fetal Bovine Serum (FBS). The concentration of FBS is about 1%-20%, about 2%-15%, about 10%-15%, about 2%, about 10%, or about 15%. In some examples, the stromal vascular cells are expanded in the presence of a serum. In some examples, the stromal vascular cells are expanded until the cells have reached about 70% confluency. In some examples the stromal vascular cells are expanded until after 1-30 passages, 6-12 passages, 8-11 passages, or 9-10 passages. In some examples, the stromal vascular cells are expanded in the presence of a basic fibroblast growth factor (bFGF). In some examples, the stromal vascular cells are expanded in the presence of a human basic fibroblast growth factor (bFGF). In some examples, the stromal vascular cells are expanded in the presence of a fish basic fibroblast growth factor (bFGF). In some examples, the stromal vascular cells are expanded in the presence of an avian basic fibroblast growth factor (bFGF). In further examples, the concentrations of the human basic fibroblast growth factor (bFGF) is about 5-100 ng/ml, about 10-80 ng/ml, about 20-60 ng/ml, or about 30-40 ng/ml.

In another example, the present disclosure describes methods of isolating adipose-derived cell lines from an animal, comprising obtaining an adipose tissue sample from an animal; collecting stromal vascular cells from the adipose tissue sample obtained; expanding the stromal vascular cells collected; and conducting clonal selection of the expanded stromal vascular cells based on the growth rates in each well. In some examples, the expanded stromal vascular cells are sorted into coated multi-well plates. It is understood by a person skilled in the art that the coating of the multi-well plates is to facilitate the attachment of the cell to the plates. The coating can be, but is not limited to poly-amino acids (e.g. poly-L-Lysine, poly-D-Lysine, poly-Ornithine), gelatin, collagen I, collagen IV, fibronectin, laminin, vitronectin, or osteopontin. It is further understandable by a person skilled in the art that the multi-well plates can be, but are not limited to 6-well plates, 12-well plates, 24-well plates, 48-well plates, or 96-well plates. In further examples, the expanded stromal vascular cells are sorted into coated multi-well plates at a cell density of about 10 cells/well. In some further examples, the cell density seeded into each well can be about 5, about 6, about 7, about 8, about 9, about 10, about 11, about 12, about 13, about 14, or about 15 cells/well. In further examples, the sorted cells in the multi-well plates are grown in the presence of a growth media for about 10 days. In some examples, the sorted cells are grown in the presence of a complete growth medium. In further examples, the complete growth medium comprises a base medium. It is understood by a person skilled in the art that the based medium can be any medium suitable for animal cell culture, including, but not limited to: Eagle's minimal essential medium (MEM), Dulbecco's modified Eagle's medium (DMEM), Iscove basic DMEM (IMDM), Roswell Park Memorial Institute medium (RPMI), 199 medium, 109 medium, Ham's F-10 medium, Ham's F-12 medium, McCoy's 5A Medium, Leibovitz's L-15 medium, advanced DMEM, Mesencult ACF Plus medium and Stempro MSC SFM CTS medium. In some examples, the sorted cells are grown in the presence of a Fetal Bovine Serum (FBS). The concentration of FBS can be about 1%-20%, about 2%-15%, about 10%-15%, about 2%, about 10%, or about 15%. In some examples, the sorted cells are grown in the presence of a serum. It is further understood by a person skilled in the art that the concentration of the serum is about 0.1%-2%, about 0.2%-1.8%, about 0.4%-1.6%, about 0.6%-1.2%, or about 0.8-1%. In some examples, the serum is obtained from animal of the same phylum. In some examples, the serum is obtained from the same species. In some further examples, the serum is a fish serum. In some other examples, the serum is an avian serum, or a porcine serum. In some examples, the sorted cells in the multi-well plates are grown in the presence of a growth media for about 15 days and cells that reach about 70% confluency are further expanded by passaging. As understood in common knowledge and by a person skilled in the art, cultured cells can be again passaged when the growth of the cells in a culture reaches about 60% confluency, about 65% confluency, about 70% confluency, about 75% confluency, about 80% confluency, or about 85% confluency. As shown in Table 1 below, for example, after about 15 days, 41 out of 1120 wells reached at least 70% confluence.

TABLE 1
Number of wells with at least 70% confluence after clonal selection

| | Pangasianodon | | |
|---|---|---|---|
| | hypophthalmus | Anguilla japonica | |
| | DMEM containing | DMEM containing | DMEM without |
| | fish serum | fish serum | fish serum |
| | Number of wells | Number of wells | Number of wells |
| At least 70% confluence | 41 | 8 | 6 |
| Total | 1120 | 192 | 192 |

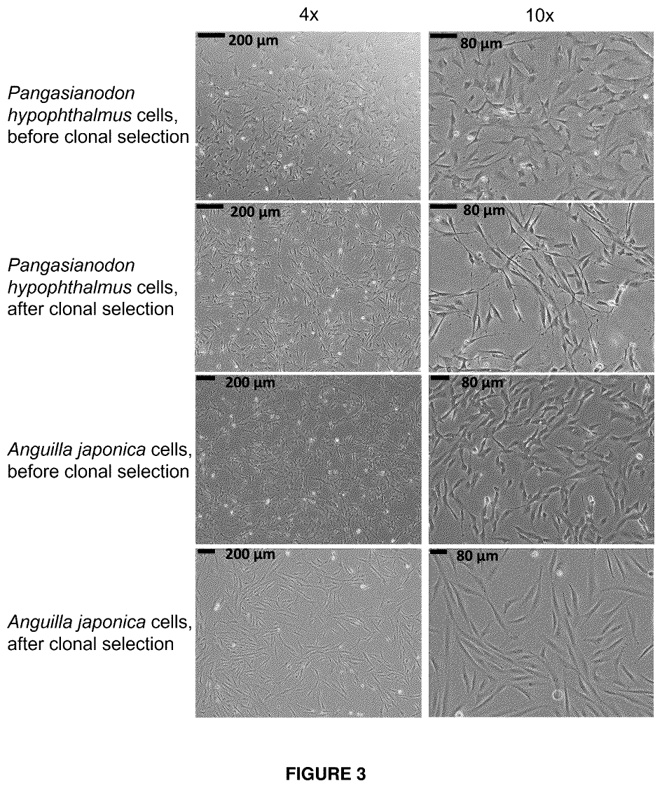

In another example, the present disclosure describes methods of isolating adipose-derived cell lines from an animal, comprising obtaining an adipose tissue sample from an animal; collecting stromal vascular cells from the adipose tissue sample obtained; expanding the stromal vascular cells collected in the presence of a serum; conducting clonal selection of the expanded stromal vascular cells based on the growth in each well; and isolating one or more adipose-derived cell lines based on the selection result. In some examples, the isolated cell lines do not enter senescence. In some examples, the isolated cell lines do not enter senescence before passage 16, passage 17, passage 18, passage 19, passage 20, passage 21, passage 22, passage 23, passage 24, passage 25, passage 26, passage 27, passage 28, passage 29, or passage 30 during expansion. In some examples, one or more adipose-derived cell lines are isolated if they reach passage 16, passage 17, passage 18, passage 19, passage 20, passage 21, passage 22, passage 23, passage 24, passage 25, passage 26, passage 27, passage 28, passage 29, or passage 30 during expansion. In some examples, the one or more isolated adipose-derived cell lines do not go into senescence after culturing for about 20 passages. In some examples, the one or more isolated adipose-derived cell lines do not go into senescence after culturing for about 16-30 passages. The isolated adipose-derived cells from one or more wells are regarded as stable cells lines. As described in FIG. 3, microscopic images show the morphology of adipose-derived cells before and after clonal selection for exemplary animal species Pangasianodon hypophthalmus and Anguilla japonica. The cells in multi-well plates displayed consistent spindle-shape morphology before and after clonal selection.

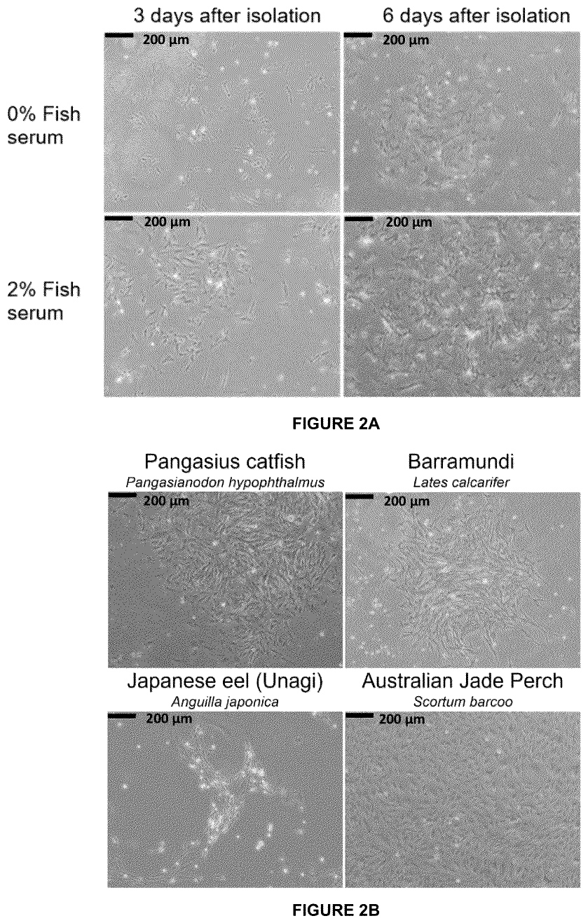

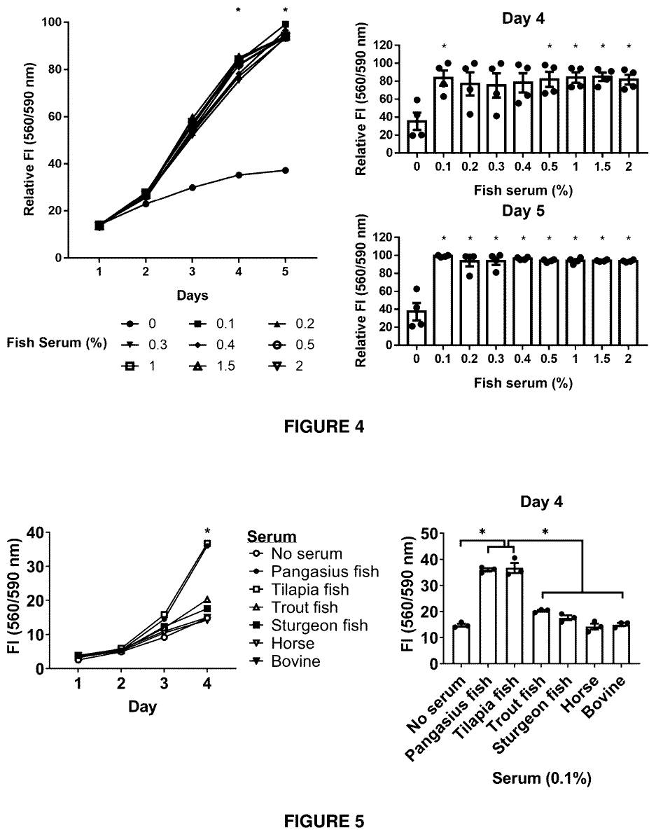

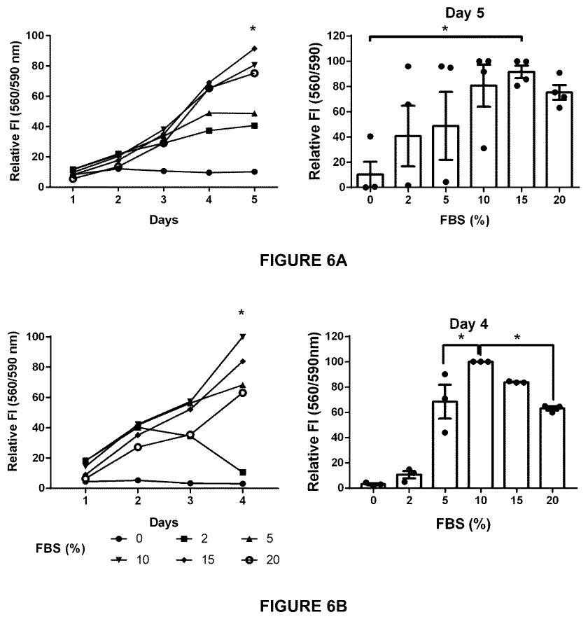

In some examples, the isolated one or more adipose-derived cell lines are cultured in the presence of a growth media in the presence of a serum. It is understood by a person skilled in the art that the concentration of the serum can be about 0.1%-2%, about 0.2%-1.8%, about 0.4%-1.6%, about 0.6%-1.2%, or about 0.8-1%. As shown in FIG. 2A and FIG. 4, for example, the isolated cell line show proliferation in the presence of serum at various concentrations, for example, about 0.1%, about 0.2%, about 0.3%, about 0.4%, about 0.5%, about 1%, about 1.5%, and about 2%. In some further examples, the serum is a fish serum. In some examples, the isolated cell lines are cultured in the presence of a serum containing complete growth medium. In further examples, the complete growth medium comprises a base medium. It is understood by a person skilled in the art that the based medium can be any medium suitable for animal cell culture, including, but not limited to: Eagle's minimal essential medium (MEM), Dulbecco's modified Eagle's medium (DMEM), Iscove basic DMEM (IMDM), Roswell Park Memorial Institute medium (RPMI), 199 medium, 109 medium, Ham's F-10 medium, Ham's F-12 medium, McCoy's 5A Medium, Leibovitz's L-15 medium, advanced DMEM, Mesencult ACF Plus medium and Stempro MSC SFM CTS medium. In some examples, the isolated cell lines are cultured in the presence of a Fetal Bovine Serum (FBS). It is understood by any person skilled in the art that concentration of FBS can be about 1%-20%, about 2%-15%, about 10%-15%, about 2%, about 10%, or about 15%. As described in FIG. 6, for example, the isolated cells are cultured in the presence of FBS at different concentrations. In other examples, the isolated one or more adipose-derived cells lines are cryopreserved.

In some examples, the present disclosure describes a method of isolating adipose-derived cell lines from an animal, comprising: (a) obtaining an adipose tissue sample from an animal; (b) collecting stromal vascular cells from the adipose tissue sample of (a); (c) expanding the stromal vascular cells of (b) in the presence of a serum; (d) conducting clonal selection of the expanded stromal vascular cells based on the growth rates in each well; and (e) isolating one or more adipose-derived cell lines based on the selection result in (d). In some examples, the tissue sample is obtained from an animal. In further examples, the animal is a poultry, a livestock, or a fish. For example, the animal is a chick, a duck, a goose, a turkey, a pigeon, or a quail. In some examples, the animal is a cattle, a sheep, a goat, a pig. In other examples, the animal includes, but is not limited to a bony fish (teleostomi) or a cartilaginous fish (chondrichthyes). In another example, the animal is Pangasianodon hypophthalmus, Anguilla japonica, Scortum barcoo, or Lates calcarifer.

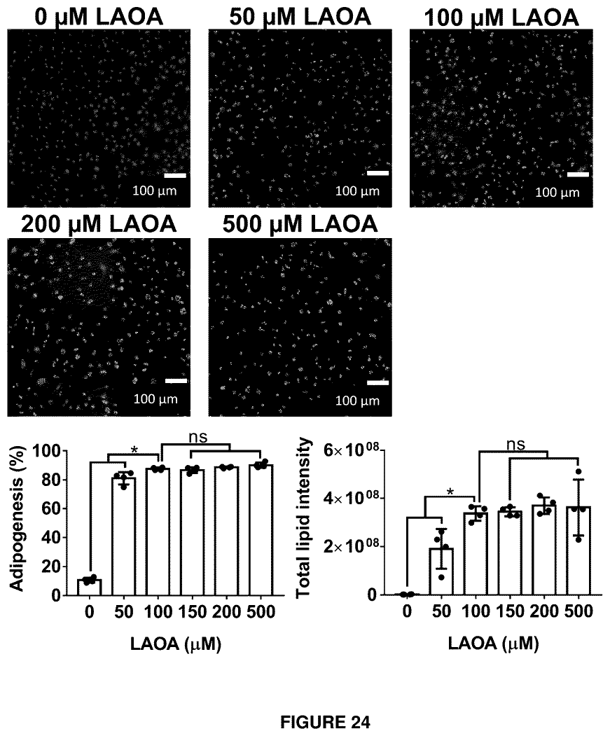

As disclosed herein, an adipogenesis induction composition is described. In one example, the present disclosure describes an adipogenesis induction composition comprising: a linoleic acid-oleic acid albumin (LAOA), an insulin, and a serum. In one example, the present disclosure describes an adipogenesis induction composition comprising: a linoleic acid-oleic acid albumin (LAOA), an insulin, a serum, and at least one, at least two, or all of 3-isobutyl-1-methylxanthine (IBMX), dexamethasone, and indomethacin. Therefore, in some examples, the adipogenesis induction composition comprises a linoleic acid-oleic acid albumin (LAOA), an insulin, a serum, and a 3-isobutyl-1-methylxanthine (IBMX). In some examples, the adipogenesis induction composition comprises a linoleic acid-oleic acid albumin (LAOA), an insulin, a serum, and a dexamethasone. In some examples, the adipogenesis induction composition comprises a linoleic acid-oleic acid albumin (LAOA), an insulin, a serum, and an indomethacin. As understood by a person skilled in the art, the term LAOA as used herein, refers to a mixture of linoleic acid and oleic acid, and albumin. In some examples, the adipogenesis induction composition comprises LAOA of the concentration of at least 10 μM, or at least 20 μM, or at least 30 μM, or at least 40 μM, or at least 50 μM. In some examples, the adipogenesis induction composition comprises LAOA of the concentration of about 50-100 μM, about 100-150 μM, or about 150-200 μM. For example, as shown in FIGS. 17 and 24, the concentration of LAOA can be about 50 PM, or about 100 μM, or about 150 μM, or about 200 μM, preferably at a concentration of about 100 μM.

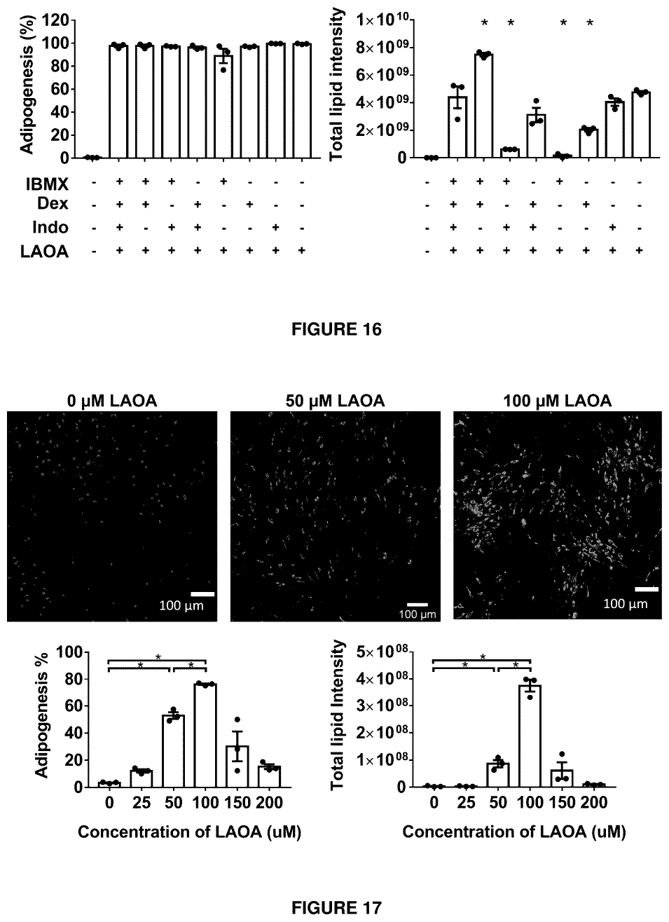

In some examples, the adipogenesis induction composition comprises a linoleic acid-oleic acid albumin (LAOA), an insulin, a serum, a 3-isobutyl-1-methylxanthine (IBMX), and a dexamethasone. In some examples, the adipogenesis induction composition comprises a linoleic acid-oleic acid albumin (LAOA), an insulin, a serum, a dexamethasone, and an indomethacin. In some examples, the adipogenesis induction composition comprises a linoleic acid-oleic acid albumin (LAOA), an insulin, a serum, an indomethacin, and a 3-isobutyl-1-methylxanthine (IBMX). In some further examples, the adipogenesis induction composition comprises a linoleic acid-oleic acid albumin (LAOA), an insulin, a serum, an indomethacin, a 3-isobutyl-1-methylxanthine (IBMX), and a dexamethasone. As shown in FIG. 16, for example, the adipogenesis induction composition as disclosed herewith supports the adipogenesis induction of the isolated exemplary adipose-derived cell lines. In some examples, the serum is a fish serum.

In some examples, the present disclosure describes an adipogenesis induction composition disclosed herewith further comprises a basic fibroblast growth factor (bFGF), and a Fetal Bovine Serum (FBS). In some examples, the present disclosure describes an adipogenesis induction composition disclosed herewith which further comprises a human basic fibroblast growth factor (bFGF), and a Fetal Bovine Serum (FBS). In some examples, the present disclosure describes an adipogenesis induction composition disclosed herewith which further comprises a fish basic fibroblast growth factor (bFGF), and a Fetal Bovine Serum (FBS). In some examples, the present disclosure describes an adipogenesis induction composition disclosed herewith which further comprises an avian basic fibroblast growth factor (bFGF), and a Fetal Bovine Serum (FBS). In further examples, the human basic fibroblast growth factor (bFGF) can be about 5-100 ng/ml, about 10-80 ng/ml, about 20-60 ng/ml, or about 30-40 ng/ml. In further examples, the concentration of FBS can be about 1%-20%, about 2%-15%, about 10%-15%, about 2%, about 10%, or about 15%. In some examples, the adipogenesis induction composition can be used in combination with a base medium. It is understood by a person skilled in the art that the base medium can be any medium suitable for animal cell culture, including, but not limited to: Eagle's minimal essential medium (MEM), Dulbecco's modified Eagle's medium (DMEM), Iscove basic DMEM (IMDM), Roswell Park Memorial Institute medium (RPMI), 199 medium, 109 medium, Ham's F-10 medium, Ham's F-12 medium, McCoy's 5A Medium, Leibovitz's L-15 medium, advanced DMEM, Mesencult ACF Plus medium and Stempro MSC SFM CTS medium. It is understood by a person skilled in the art that the composition can be used in combination with other commonly used supplements in a cell culture, for example, amino acids, vitamins, inorganic salts, glucose, growth factors, hormones, and attachment factors. It is also understood that the adipogenesis induction composition disclosed herewith maintains a suitable pH and osmolality for cell growth. For a person skilled in the art, it is easily understood that the temperature, humidity and gaseous atmosphere of cell culture depends on the type of cells, and can be optimized routinely.

As disclosed herein, a method of obtaining adipocytes from an animal is described. In one example, the present disclosure describes a method of obtaining adipocytes from an animal, comprising isolating adipose-derived cells from a tissue sample of the animal. For example, the tissue samples can be an adipose tissue. In some examples, the adipose tissue sample obtained herein can be, but is not limited to a fat tissue, a visceral fat, a subcutaneous fat, a bone marrow fat, a mesenteric fat, or an intramuscular/intermuscular fat comprised in a muscle. The method of isolating adipose-derived cells from a tissue sample can be, for example, the method as disclosed earlier. A person skilled in the art would be able to appreciate that an isolated adipose-derived cell obtained using other methods would be applicable to the method of obtaining adipocytes from an animal as disclosed herewith. In some examples, the tissue sample is obtained from an animal. In further examples, the animal is a poultry, a livestock, or a fish. For example, the animal is a chick, a duck, a goose, a turkey, a pigeon, or a quail. In some examples, the animal is a cattle, a sheep, a goat, a pig. In other examples, the animal includes, but is not limited to a bony fish (teleostomi) or a cartilaginous fish (chondrichthyes). In another example, the animal is Pangasianodon hypophthalmus, Anguilla japonica, Scortum barcoo, or Lates calcarifer.

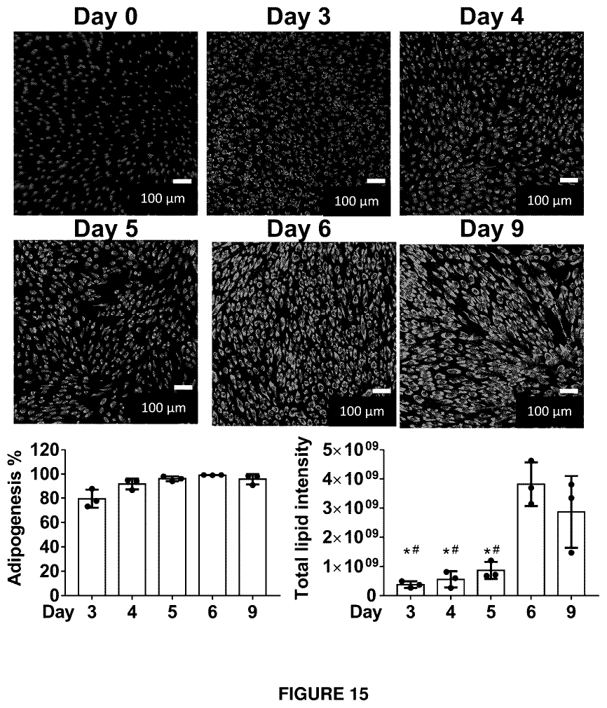

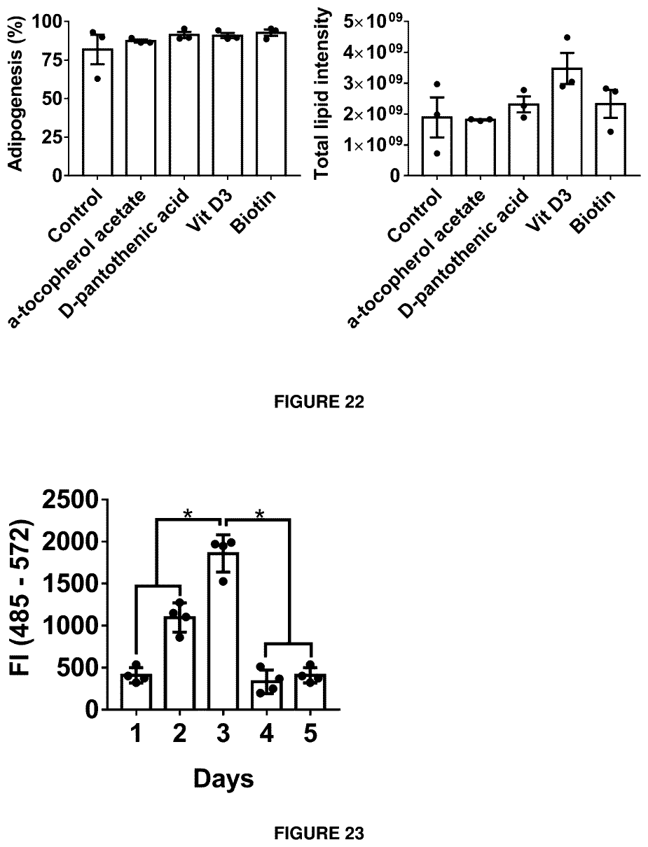

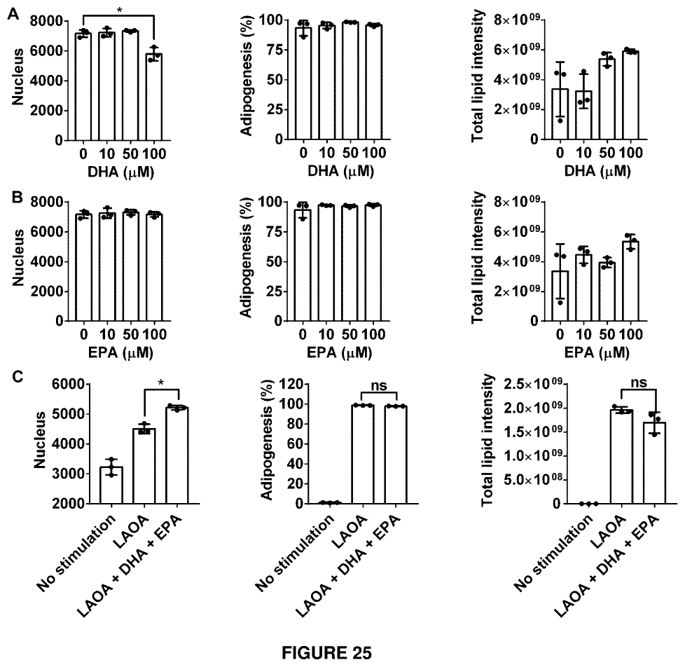

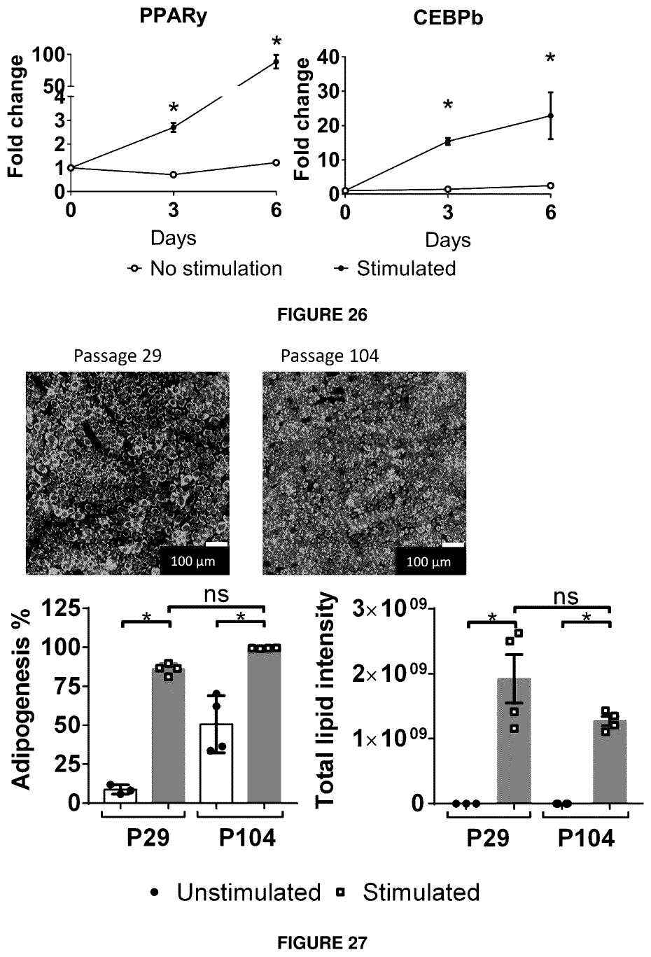

In some examples, the present disclosure describes a method of obtaining adipocytes from an animal, comprising isolating adipose-derived cells from a tissue sample of the animal; and culturing the adipose-derived cells in the presence of the adipogenesis induction composition as disclosed herein. In some examples, the adipose-derived cells are cultured in the additional presence of any one or more of vitamins or omega-3 fatty acids. For example, FIGS. 22 and 25 shows the addition of vitamin D3, vitamin C, or omega-3 fatty acids (including DHA, EPA, for example) has no adverse effects on the adipogenesis induction efficiency. In further examples, adipose-derived cells are cultured in the presence of the adipogenesis induction composition as disclosed herein for at least about 3 days, or about 6 days, or about 9 days. In some examples, the adipogenesis induction is at least about 6 days. As demonstrated in FIG. 15, for example, the percentage of cells undergoing adipogenesis is about 80% from day 3. In some examples, the adipose-derived cells are cultured in the presence of the adipogenesis induction composition as disclosed herein, wherein the serum, 3-isobutyl-1-methylxanthine (IBMX), dexamethasone, and indomethacin can be optionally removed after about 3 days, about 4 days, about 5 days, about 6 days of culturing.

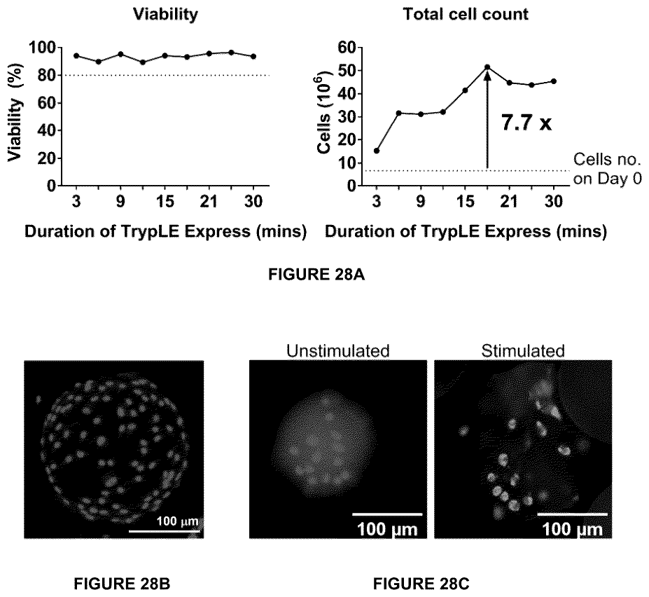

In some examples, the present disclosure describes a method of obtaining adipocytes from an animal, comprising isolating adipose-derived cells from a tissue sample of the animal; and culturing the adipose-derived cells in the presence of the adipogenesis induction composition as disclosed herewith; and obtaining adipocytes from the cell culture. In some examples, the method of obtaining adipocytes from an animal as disclosed herein is carried out in a large-scale cell culture. In some examples, the method of obtaining adipocytes from an animal as disclosed herein is carried out in a bioreactor. In some examples, the bioreactor comprises a suspension culture system. In some examples, the suspension culture system is a microcarrier. For example, as shown in FIG. 28, the adipocytes are obtained using bioreactors comprising microcarriers for adherence of cultured cells.

In some examples, the present disclosure describes a method of obtaining adipocytes from an animal, comprising isolating adipose-derived cells from a tissue sample of the animal; and culturing the adipose-derived cells in the presence of the adipogenesis induction composition as disclosed herewith; and obtaining adipocytes from the cell culture. In some examples, the step of isolating adipose-derived cells from a tissue sample of the animal is based on the method as disclosed herein.

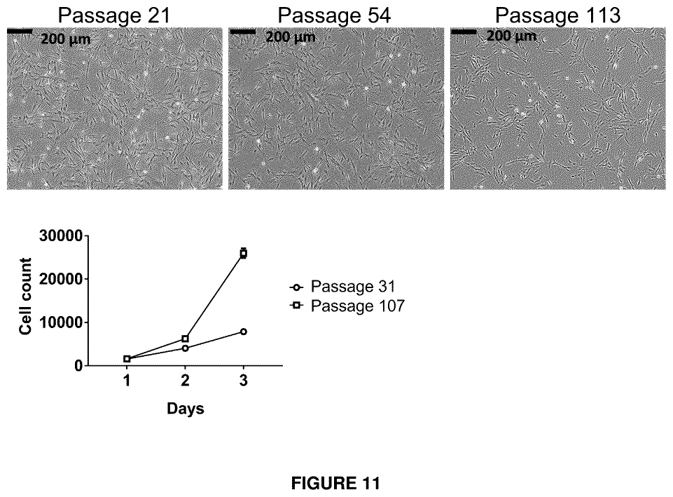

As disclosed herein, an adipose-derived cell line is described. In one example, the present disclosure refers to an adipose-derived cell line obtained or obtainable by the method disclosed herein. In some examples, the present disclosure refers to an adipose-derived cell line obtained or obtainable by the method of isolating adipose-derived cell lines from an animal, comprising: (a) obtaining an adipose tissue sample from an animal; (b) collecting stromal vascular cells from the adipose tissue sample of (a); (c) expanding the stromal vascular cells of (b); (d) conducting clonal selection of the expanded stromal vascular cells based on the growth rates in each well; and (e) isolating one or more adipose-derived cell lines based on the selection result in (d). In another examples, the present disclosure refers to an adipose-derived cell line obtained or obtainable by the method disclosed herein, wherein the cell line is characterized by not entering senescence state after at least 21 passages. As demonstrated in FIG. 11 with an exemplary adipose-derived cell line, the cell line stably proliferates in cell culture beyond 21 passages, to more than one hundred passages. In some examples, the cell line is characterized by not entering senescence state after at least 16 passages, at least 21 passages, at least 30 passages, at least 50 passages, at least 80 passages, or at least 100 passages. It is also shown in FIG. 27, for example, that for cells undergoing at least 29 passages, or at least 104 passages, the exemplary cell line still shows capability of undergoing adipogenesis induction using the methods disclosed herein.

In some examples, the adipose-derived cell lines can be: Ph9F-1x (Deposition accession number: CBA20220039) or Aj1C-1x. The exemplary cell line Ph9F-1x disclosed herein is deposited under Budapest Treaty in CellBank Australia on 16 Jun. 2022, with deposition accession number of CBA20220039 by one of the inventors, Lamony Chew from Institute of Molecular and Cell Biology, which is one of the institutes organized under Agency for Science, Technology and Research (A*STAR). The deposited cell line is tested to be viable by CellBank Australia on 20 Jun. 2022.

As disclosed herein, a cell line Ph9F-1x (Deposition accession number: CBA20220039) is described. In some examples, the cell line is an adipose-derived cell line. In some examples, the cell line Ph9F-1x is characterized by not entering senescence state after at least 16 passages, at least 21 passages, at least 30 passages, at least 50 passages, at least 80 passages, or at least 100 passages. In some examples, the cell line can be obtained, or is obtainable by the method of isolating adipose-derived cell lines as disclosed herein. In some examples, the cell line can be used to induce adipogenesis using the adipogenesis induction composition as disclosed herein. In some further examples, the cell line can be used for obtaining adipocyte from an animal, based on the methods as disclosed herein starting from step (b) culturing the adipose-derived cells in the presence of the adipogenesis induction composition as disclosed herein.

As disclosed herein, a cell line Aj1C-1x is described. In some examples, the cell line is an adipose-derived cell line. In some examples, the cell line Aj1C-1x is characterized by not entering senescence state after at least 16 passages, at least 21 passages, at least 30 passages, at least 50 passages, at least 80 passages, or at least 100 passages. In some examples, the cell line can be obtained, or is obtainable by the method of isolating adipose-derived cell lines as disclosed herein. In some examples, the cell line can be used to induce adipogenesis using the adipogenesis induction composition as disclosed herein. In some further examples, the cell line can be used for obtaining adipocyte from an animal, based on the methods as disclosed herein starting from step (b) culturing the adipose-derived cells in the presence of the adipogenesis induction composition as disclosed herein.

In some examples, the adipose-derived cell lines disclosed herein undergo exponential expansion with unlimited space and nutrients. In some examples, the adipose-derived cell lines disclosed herein undergo exponential expansion within the first 72 hours. In some examples, the doubling time is less than about 48 hours. In some examples, the doubling time is about 10-48 hours. In further examples, the adipose-derived cell lines disclosed herein have a doubling time of about 12-48 hours. Example 8 demonstrates, for example, the exemplary cell lines have a doubling time measured to be about 13.7 and about 31.2 hours, respectively.

In some examples, the adipose-derived cell lines disclosed herein are cultured in a growth media as disclosed herein, or are cryopreserved. In some examples, the adipose-derived cell lines disclosed herein contains no mycoplasma contamination. In some examples, adipose-derived cell lines are obtained or obtainable from an animal. In further examples, the animal is a poultry, a livestock, or a fish. For example, the animal can be a chick, a duck, a goose, a turkey, a pigeon, or a quail. In some examples, the animal can be a cattle, a sheep, a goat, a pig. In other examples, the animal can be, but is not limited to a bony fish (teleostomi) or a cartilaginous fish (chondrichthyes). In another example, the animal can be Pangasianodon hypophthalmus, Anguilla japonica, Scortum barcoo, or Lates calcarifer.

As disclosed herein, a kit for adipogenesis induction of adipose-derived animal cells in vitro is described. In one example, the kit for adipogenesis induction of adipose-derived animal cells in vitro, comprising the adipogenesis induction composition as disclosed herein and the adipose-derived cell lines as disclosed herein. In another example, the kit for adipogenesis induction of adipose-derived animal cells in vitro, comprising the adipogenesis induction composition as disclosed herein and an isolated adipose-derived cell line obtained according to the methods disclosed herein.

As disclosed herein, an adipose cell produced by the methods as disclosed herein is described. In some examples, the adipose cell is an adipocyte.

As disclosed herein, a food product comprising the cell line as disclosed herein is described. In one example, the food product comprises the adipose cell as disclosed herein. In some examples, the food product comprises a meat. In some examples, the food product is a cultivated meat. It is understood by a person skilled in the art that the food product comprising the cell line as disclosed herein, or the adipose cell as disclosed herein is manufactured by combining with other cell types, for example, matured muscle cells, and 3-dimensional scaffolds of editable food grade material. In some examples, the food product comprises fish meat.

As disclosed herein, a lipid composition is described. In one example, the lipid composition is obtained from the adipose-derived cell line as disclosed herein. In another example the lipid composition is obtained from the adipose cells as disclosed herein. In some examples, the lipid composition is in liquid form or solid form under room temperature. In some examples, the lipid composition is a fish oil.

As disclosed herein, use of the lipid composition disclosed herein is described. In some examples, the fat undergoes industrial processing prior to use. In some examples, the fat is used in the manufacture of cosmetic products. In some examples, the fat is used as a nutritional supplement. In some examples, the fat is used as food additives.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The disclosure has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein. Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Animals

Patin fish, Pangasianodon hypophthalmus, of about 0.7-1.0 kg were obtained from a local fish farm, Khaiseng Trading & Fish Farm Pte. Ltd., Singapore. Japanese eel, Anguilla japonica, of about 0.1-0.2 kg were obtained from Man Man Japanese Unagi Restaurant, Singapore. Barramundi, Lates calcarifer, and Australian jade perch, Scortum barcoo of about 0.7-1.0 kg were obtained from another local fish farm, Apollo Marine Seafood, Singapore. The fishes were sacrificed by immersion in tricaine methane-sulfonate solution, followed by decapitation. After euthanasia, around 0.6-1.5 g of mesenteric fats were excised for stem cell isolation. In some cases, muscle containing intramuscular or intermuscular fat was used for cell isolation. Further, around 10 ml of blood was collected via dorsal venous puncture and stored in 4° C. for 18 hours. Serum was collected after centrifugation at 2,000×g for 15 minutes. The serum was subjected to heat treatment at 56° C. for 30 minutes and stored at −30° C. until further processing. All procedures involving live animal handling were performed as approved by the Institutional Animal Care and Use Committee (IACUC) of A*STAR, Singapore (IACUC no. #201570).

### Growth Media

All cell culture reagents and chemicals were purchased from Thermo Fisher Scientific, USA unless otherwise stated. Complete growth media comprised of high glucose DMEM, 20% heat-inactivated FBS, 2% heat-inactivated fish serum, 300 U/ml of penicillin, 300 μg/mL of streptomycin, and 5 ng/ml bFGF. Advanced DMEM was supplemented with 2% heat-inactivated FBS, 0.1% heat-treated fish serum, 5 ng/ml bFGF, and 2 mM L-glutamine. Leibovitz's L-15 medium was supplemented with 15% heat-inactivated FBS, 0.1% heat-treated fish serum, and 5 ng/ml bFGF. Mesencult ACF Plus (Stemcell Technologies) and Stempro MSC SFM CTS media were supplemented with 0.1% heat-treated fish serum, and 2 mM L-glutamine.

### Isolation of Fish Adipose-Derived Cells

Stromal vascular fractions were isolated from the excised mesenteric fats by enzymatic digestion. Briefly, the fat or muscle tissues were submerged in equal volume (microliter per gram of weight of fat) of Hanks' Balanced Salt Solution (HBSS) and minced using a pair of sterilized scissors. The minced fat tissues were digested in equal volume (ml per g weight of fat) of HBSS containing 1 mg/ml of collagenase type IV (Worthington Biochemical Corporation, NJ, USA) and 5 mM calcium chloride in 37° C. for 1 hour. Alternatively, the minced fat tissues were digested in equal volume (microliter per gram of weight of fat) of HBSS containing 0.5 mg/ml of collagenase type IV, 2.4 mg/ml of dispase II, and and 2.5 mM calcium chloride in in 37° C. for 1 hour. The digested fat tissue samples were passed through 100 μm nylon filters and subjected to centrifugation at 400×g for 5 minutes. The cell pellets were resuspended in Ammonium-Chloride-Potassium (ACK) lysing buffer for 2 minutes and passed through 40 μm nylon filters. The cells were resuspended in complete growth media and grown on gelatin-coated (Stemcell Technologies) 6-well plate at 28° C. in a humidified atmosphere containing 5% CO2. Cell culture media were replaced every 2 days and cells were passaged by incubating with 0.05% Trypsin-Ethylenediaminetetraacetic acid (EDTA) or Tryple Express at 28° C. for 2 minutes upon reaching 70% confluent.

Alternatively, adipose-derived cells were isolated from the excised mesenteric fats by explant culture. The fat tissues were minced into small pieces of about 4-5 mm in diameter and resuspended in complete growth media as described above. Cell culture media were replaced every 7 days until cells migrated out of the tissues and reached 30-40% confluent. Thereafter, cell culture media were replaced every 2 days until 70% confluent.

### Establishment of Cell Lines

Cells were grown until the 9th passage and sorted into gelatin-coated 96-well flat-bottom black plates at cell density of 10 cells/well using BD FACSAria II Cell Sorter (BD Biosciences, USA). The cells were grown in complete growth media and incubated at 28° C. in a humidified atmosphere containing 5% CO2. After 10 days, the wells with at least 70% confluent were expanded. The cells that reached passage 20 were regarded as cell lines and cryopreserved in liquid nitrogen using Bambanker freezing medium.

### Measurement of Cell Proliferation

Cells were resuspended at cell density of 50,000 cells/ml of growth medium containing titrating concentration of FBS or fish serum. The cells were seeded into gelatin-coated 96-well flat-bottom black plates and incubated at 28° C. in a humidified atmosphere containing 5% CO2. Cell proliferation of the cells were assessed over 5 days using CellTiter-Blue Assay (Promega, USA) as per manufacturer's instruction. Briefly, on each day, CellTiter-Blue reagent was added to the cells and incubated at 28° C. After 2 hours, the fluorescence intensity was measured at excitation/emission wavelength of 560/590 using Tecan Infinite M200 Pro.

Detection of mycoplasma Contamination

Cells were grown in T75 flask for 2 days until 70% confluence. Cell culture supernatant were collected, and the presence of mycoplasma was detected using MycoAlert™ PLUS mycoplasma detection kit (Lonza) as per manufacturer's instructions. Luminescence was measured using Tecan Infinite M1000 plate reader.

### Measurement of Cell Population Doubling Time

Cells were resuspended in complete growth media and added into gelatin-coated 96-well flat-bottom black plates at cell density of 1000 or 1500 cells per well. The cells were incubated at 28° C. in a humidified atmosphere containing 5% CO2 for 3 days. On each day, cell nuclei were stained with CyQUANT cell proliferation assay as per manufacturers' instruction. After 1 hour of incubation at 28° C., the cells imaged using N-STORM/TIRF microscope at SBIC-Nikon Imaging Centre at Biopolis, Singapore and the number of cells were determined using NIS-Element software (Nikon, Tokyo, Japan).

### Adipogenesis of Fish Adipose-Derived Cell Lines

Adipogenesis of adipose-derived cells were induced. The cells were resuspended at cell density of 100,000 cells/ml of complete growth medium and seeded at 100 μl/well into gelatin-coated 96-well flat-bottom black plates. The cells were incubated at 28° C. in a humidified atmosphere containing 5% CO2. After 2 days, the cells were washed with HBSS thrice and adipogenesis was initiated by inducing the cells with high glucose Dulbecco's Modified Eagle Medium (DMEM), 15% heat-inactivated Fetal Bovine Serum (FBS), 0.1% heat-inactivated fish serum, 300 U/ml of penicillin, 300 μg/mL of streptomycin, and adipogenic induction cocktail 1, containing 500 μM of 3-isobutyl-1-methylxanthine (IBMX, Sigma-Aldrich), 1 μM of dexamethasone, 167 nM of insulin, and 100 μM of indomethacin (Sigma-Aldrich). After 3 and 6 days of adipogenic induction, the cells were induced with DMEM, 15% FBS, 0.1% fish serum, 300 U/ml of penicillin, 300 μg/mL of streptomycin, and adipogenic induction cocktail 2, containing 167 nM of insulin. Linoleic acid-oleic acid albumin (LAOA, Sigma-Aldrich), docosahexaenoic acid (DHA, Cayman Chemical), eicosapentaenoic acid (EPA, Cayman Chemical), oleic acid albumin (OAA, Sigma Aldrich), and linoleic acid albumin (LAA, Sigma Aldrich) can also be added to the adipogenic induction cocktail, if required. After 9 days of adipogenic induction, the cells were stained with AdipoRed assay reagent (Lonza, USA), fixed with 4% paraformaldehyde, and counterstained with Hoechst 33342 as per manufactures' instructions. The cells were imaged using N-STORM/TIRF microscope and analysed using NIS-Element software.

### Gene Expression Analysis

Total RNA was extracted using RNeasy Plus Mini Kit (Qiagen) and reverse transcribed to cDNA using SuperScript™ IV First-Strand Synthesis System (Thermo Fisher Scientific). The expression of various genes involved in adipogenic differentiation (PPAR-γ, C/EBPα, C/EBPβ, FAS, HSLα, HSLβ, LPL) were examined using SsoAdvanced Universal SYBR Green Supermix (Bio-rad) on CFX96 real-time PCR detection system. Relative gene expression was normalized to β-actin and calculated using 2-ΔΔCT method. The primers were designed using Primer3 version 4.1.0, based on the predicted sequences of Pangasianodon hypophthalmus on NCBI database.

### Measurement of Omega-3 Fatty Acids

Omega-3 fatty acids were extracted and measured as described by the Duke-National University of Singapore (NUS) Metabolomics Facility with slight modifications. Cells were washed with cold Phosphate-buffered saline (PBS) and scraped in cold 0.1% formic acid. The cells were then mixed on vortex at 4° C. for 30 minutes. Protein concentration of the cell extract was measured using Pierce reducing agent compatible microplate BCA protein assay kit (Thermo Fisher Scientific, USA) as per manufacturer's instruction. The cell extract was mixed with equal volume of acetonitrile and sent to the Duke-NUS Metabolomics Facility for mass spectrometry analysis.

### Growth and Adipogenesis of Fish Cell Lines on Cytodex 1 Microcarriers in Spinner Flask

Cell lines were grown on Cytodex 1 microcarriers (Cytiva Life Sciences, USA) as per manufacturer's instructions. Briefly, 6.67×10⁶ cells, 0.2 gram of microcarriers, and 40 ml of complete growth media were mixed in a 125 ml spinner flask and stirred at 20 revolutions per minute (rpm). After 2 hours, the stirring rate was increased to 40 rpm. After 24 hours, 30 ml of complete growth media were added to the flasks. To replenish the growth medium, the cell-microcarrier aggregates were allowed to settle for 2 minutes and 50% of the spent medium were replaced with fresh medium every 2 days. To induce adipogenic differentiation of cell lines, the cell-microcarrier aggregates were treated with Essential 6 media containing fish serum, adipogenesis induction cocktail and LAOA for 6 days.

To determine the cell number, the cell-microcarrier aggregates were washed with 70 ml of PBS thrice, treated 70 ml of Tryple Express, and stirred at 150 rpm at 28° C. The cells and microcarriers were separated using 70 μm cell strainer (SPL Life Sciences, South Korea) and counted with Trypan Blue stain (Thermo Fisher Scientific, USA). To visualize the adherence of the cells onto the microcarriers, cells were stained with Hoechst 33342 as per manufactures' instructions and imaged using Nikon A1R+si confocal microscope at SBIC-Nikon Imaging Centre at Biopolis.

### Statistical Analysis

All data are presented as mean±SEM, unless otherwise stated. Significant differences (P<0.05) of each variable were first determined using the one-way analyses of variance (ANOVA), followed by Tukey post-hoc test. All analyses were performed using GraphPad Prism 5.03 (GraphPad Software, Inc., San Diego, CA).

### EXAMPLES

### Example 1

### Isolation of Adipose-Derived Primary Cells

The adipose tissues of Pangasianodon hypophthalmus were digested in collagenase type IV and grown in complete DMEM in the absence or presence of 2% fish serum. The results indicate successful isolation and growth of Pangasianodon hypophthalmus adipose-derived cells. Adipose-derived cells from barramundi (Lates calcarifer), Japanese eel (Anguilla japonica), and Australian jade perch (Scortum barcoo) were also isolated and grown successfully using the same method (FIG. 2B). As compared to the absence of fish serum, the presence of fish serum in the growth media resulted in higher cell density of freshly isolated cells after 3 and 6 days of culture (FIG. 2A). The results suggest that fish serum contributes to the survival of newly isolated Pangasianodon hypophthalmus adipose-derived cells.

### Example 2

### Clonal Selection

As adipose-derived primary cells consist of heterogeneous populations of cells, the cells of Pangasianodon hypophthalmus were expanded by 10 passages and split at 10 cells per well into 96-well plates containing DMEM with 2% fish serum. After 15 days, 41 out of 1120 wells reached at least 70% confluence (Table 1). The cells in these wells displayed the same spindle-shape morphology before and after clonal selection (FIG. 3). These cells were expanded and cryopreserved for future experiments. The cells that were able to be expanded by more than 20 passages were regarded as fish adipose-derived cell lines.

The cells of Anguilla japonica were expanded by 6 passages in DMEM containing 2% fish serum. The cells were then split at 1 cell per well into 96-well plates containing DMEM with or without 2% fish serum. After 13 days, 8 out of 192 wells in DMEM with 2% fish serum reached at least 70% confluence and 6 out of 192 wells in DMEM without fish serum reached at least 70% confluence (Table 1). The cells in these wells also displayed the same spindle-shape morphology before and after clonal selection (FIG. 3). These cells were expanded and cryopreserved for future experiments.

### Example 3

### Growth of Adipose-Derived Cells at Low Concentration of Serum

As the availability and cost of serum are the limitations of growing adipose-derived cells or cell lines, Pangasianodon hypophthalmus cell lines were grown in titrating concentration of fish serum to determine the minimum concentration of fish serum that can support the growth of fish cell lines. As compared to the absence (0%) of fish serum, Pangasianodon hypophthalmus cell lines that were grown in the presence of at least 0.1% fish serum displayed significantly higher fluorescence intensity after 4 and 5 days (FIG. 4). The results indicate that the presence of at least 0.1% fish serum in the growth media enhances the proliferation of fish cell lines. There was no significant difference in the proliferation of fish cell lines that were grown in the presence of 0.1-2% fish serum. Thus, the data suggest that 0.1% of fish serum would be required to support the growth of Pangasianodon hypophthalmus cell lines

### Example 4

### Growth of Adipose-Derived Cells in the Presence of Autologous Sera

The exemplary cell line Ph9F-1x is grown in the presence of Pangasius and tilapia fish sera as compared to trout fish, sturgeon fish, horse and bovine sera. The growth rates of the adipose-derived cells are compared. Exemplary cell line Ph9F-1x is shown to proliferate at a faster rate in the presence of Pangasius and tilapia fish sera as compared to trout fish, sturgeon fish, horse and bovine sera, indicating that autologous serum or serum from an animal of the same phylum play a role in cell proliferation during cell isolation or maintenance.

### Example 5

### Growth of Adipose-Derived Cells Requires FBS

Next, concentration of FBS that supports the growth of fish cell lines was investigated. Pangasianodon hypophthalmus and Anguilla japonica cell lines did not proliferate in the absence of FBS (FIG. 5). In contrast, Pangasianodon hypophthalmus and Anguilla japonica cell lines proliferated in the presence of FBS, indicating that FBS is required to support the growth of fish cell lines.

Pangasianodon hypophthalmus cell lines showed significantly higher proliferation rate in the presence of 15% FBS (91.5±4.9%) as compared the cell lines grown in the absence of FBS (10.2±10.1%) after 5 days (FIG. 5A). There was no significant difference in the proliferation rate of the cell lines that were grown in the presence of other concentrations of FBS.

Anguilla japonica cell lines showed significantly higher proliferation rate in the presence of 10% FBS (100%) as compared the cell lines grown in 20% FBS (63.1±2.8%) and 5% FBS (68.3±23.2%) after 4 days (FIG. 5B). There was no significant difference in the proliferation of the cell lines that were grown in the presence of 10% FBS as compared to 15% FBS.

### Example 6

### Identification of Fast- and Slow-Growing Cell Lines

Cell growth rate is one of the most important factors during the production of cultivated meat. To characterize the growth rate of different fish adipose-derived cell lines, the growth rate of 8 Pangasianodon hypophthalmus cell lines was examined over 5 days. The proliferation assay revealed that Ph9F-1x (denoted by open squares) proliferate faster as compared to the other cell lines (FIG. 6).

The growth rate of 5 Anguilla japonica cell lines was also examined over 4 days. The proliferation assay revealed that Aj1C-1x (denoted by open squares) proliferate faster as compared to the other cell lines (FIG. 6). Both Ph9F-1x and Aj1C-1x were selected for adipogenesis studies.

### Example 7

### Detection of Mycoplasma Contamination in Cell Lines

Mycoplasma infection is a common problem during cell culture, and it is known to reduce the growth rate of cell lines. To ensure that mycoplasma contamination did not occur in the isolated adipose-derived cell lines, the culture supernatant of fast-growing fish cell lines, Ph9F-1x and Aj1C-1x, and other slow growing clones were tested for the presence of mycoplasma. The mycoplasma assay revealed that the relative luminescence units of the 9 cell culture supernatants were below 1.0, indicating that the isolated adipose-derived cell lines were not infected by mycoplasma. Further, the difference in growth rate between two isolated cell lines Ph9F-1x, Aj1C-1x and other clones was not due to mycoplasma contamination.

### Example 8

### Identifying Compatible Growth Media for Cell Growth

Apart from DMEM, many studies have used Mesencult ACF Plus and Stempro MSC SFM CTS media to support the growth of human adipose-derived stem cells and Leibovitz's L-15 growth media to support the growth of cultured cells. To determine the most suitable growth media that can support the growth of isolated adipose-derived cell lines, Pangasianodon hypophthalmus cell lines were grown in different growth media. Cell proliferation assay revealed that Pangasianodon hypophthalmus adipose-derived cell lines that were grown in DMEM and advanced DMEM showed significantly higher proliferation rate as compared to Leibovitz's L-15, Mesencult ACF Plus, and Stempro MSC SFM CTS. There was no significant difference in the proliferation of fish cell lines that were grown in DMEM and advanced DMEM. Downstream experiments were performed using DMEM.

### Example 9

### Estimation of Cell Population Doubling Time

Short population doubling time is an important feature of cell-based meat as it determines the scalability and cost-effectiveness of product. To determine the growth rate of the isolated cell lines, a fast-growing Pangasianodon hypophthalmus cell line, Ph9F-1x, and a Anguilla japonica cell line, Aj1C-1x, were seeded at 1000 and 1500 cells per well respectively, and the number of nuclei were measured every 24 hours. Ph9F-1x and Aj1C-1x were shown to expand exponentially over 72 hours with an average population doubling time of 13.7 and 31.2 hours respectively. The approximate doubling time of other cell lines falls under 48 hours, while most cell lines ranged from 10 to 48 hours.

### Example 10

### Cell Morphology of Cell Line During Early and Late Passages

Primary cells usually enter senescence state after 10-15 passages as shown by reduction in growth rate and changes in cell morphology from spindle shape to enlarged, flattened, and irregular shape. The number of possible passages and cell divisions are potential concerns in cell-based meat as it limits scalability or large-scale production of cell-based meat. Therefore, Ph9F-1x was grown in complete media and subcultivated at 1:5 ratio upon reaching 80% confluence. The cell line was able to expand to more than 113 passages, which is equivalent to approximately 226 population doubling levels. Bright field images also revealed that Ph9F-1x retained its spindle shape morphology over different passages. Further cell proliferation assay revealed that the growth rate of Ph9F-1x at passage 107 was faster than Ph9F-1x at passage 31. These data suggest that Ph9F-1x did not enter senescence state and it can potentially be used for large scale production of cell-based meat.

### Example 11

### Induction of Adipogenesis Using Adipogenesis Induction Media and Linoleic Acid-Oleic Acid Albumin (LAOA)

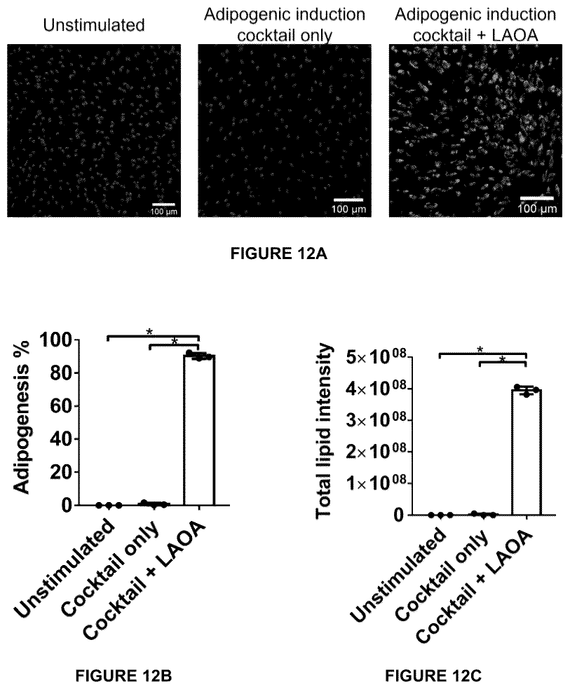

The most important feature of the adipose-derived cell lines is their ability to differentiate into mature adipocytes in a controlled manner. Adipogenic induction cocktails, containing IBMX, dexamethasone, insulin, and indomethacin, are commonly used to differentiate human or mouse adipose-derived stem cells into mature adipocytes. Here, Ph9F-1x was treated with adipogenic induction cocktail 1 for 3 days, followed by adipogenic induction cocktail 2 for 6 days. The staining results revealed that adipogenic induction cocktail did not significantly increase the number of cells expressing lipids (FIGS. 12A and 12B) and total lipid intensity (FIGS. 12A and 12C) as compared to unstimulated cells.

To enhance adipogenesis, fish adipose-derived cell lines were treated with adipogenic induction cocktails with 100 μM of linoleic acid-oleic acid albumin (LAOA). The staining results revealed that the number of cells expressing lipids and relative lipid accumulation were significantly higher after treatment with adipogenic induction cocktail with LAOA as compared to adipogenic induction cocktail without LAOA, and unstimulated control (FIG. 12).

### Example 12

### Adipogenic Differentiation of Other Isolated Cell Lines

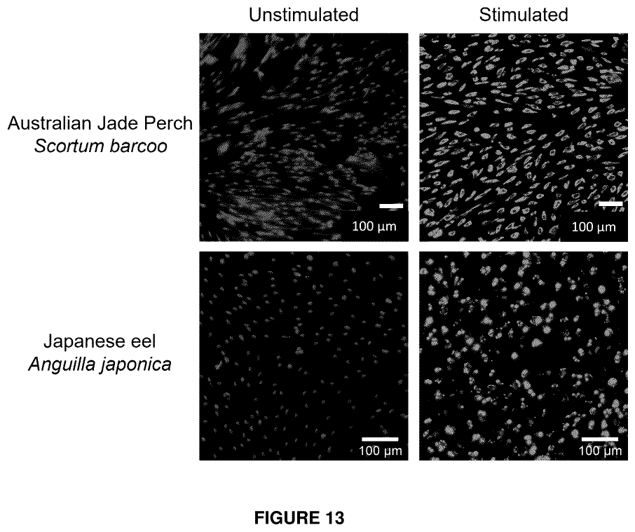

As a proof of concept, cell lines from Anguilla japonica and Scortum barcoo were also treated with the adipogenic induction cocktails with LAOA over 9 days as described earlier. The staining results revealed that both Anguilla japonica and Scortum barcoo cell lines differentiated into matured adipocytes as shown by the increase in lipid accumulation (FIG. 13).

### Example 13

### Culturing Isolated Cell Lines in FBS-Free Media

The usage of FBS in cell-based meat raises concerns regarding sustainability, cost-effectiveness, lot-to-lot variations, and animal cruelty. FBS-free media, for example, Essential 6 media, is a feeder-free, xeno-free, and serum-free media that supports the growth or differentiation of stem cells. Here, we compared the adipogenesis potential of Ph9F-1x in FBS-containing DMEM and FBS-free Essential basal media as illustrated in FIG. 24A. Previously, DMEM was used as the exemplary basal media, which requires adipogenesis induction cocktail and LAOA to induce adipogenesis. This adipogenesis induction cocktail contains 167 nM of insulin. However, Essential 6 basal media already contains 3.34 μM of insulin. Thus, insulin was not added into Essential 6 basal media during adipogenesis.

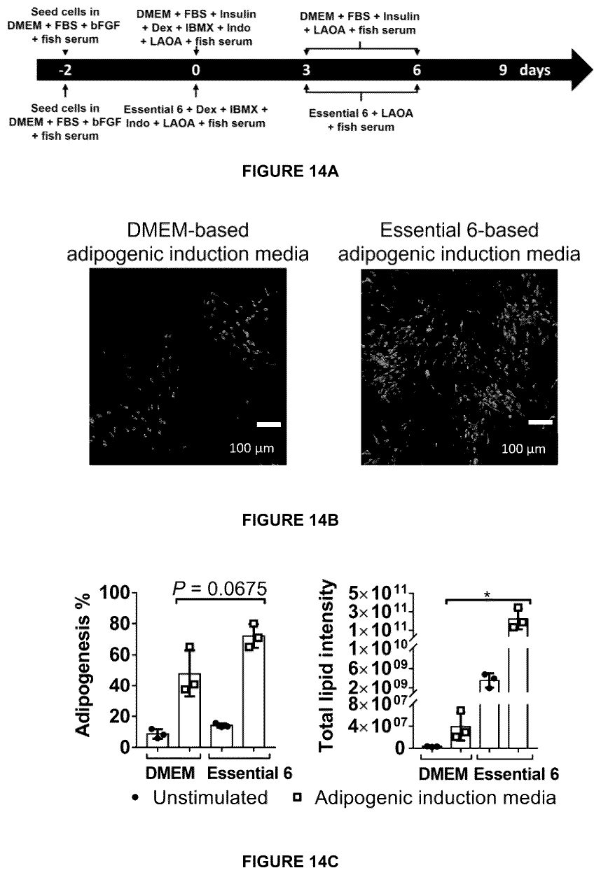

The staining results showed that Essential 6-based adipogenic induction media enhanced the number of cells expressing lipids as compared to DMEM-based adipogenic induction media, albeit with borderline significance of P=0.0675 (FIGS. 14B and 14C). Most importantly, Essential 6-based adipogenic induction media significantly enhanced the total lipid intensity as compared to DMEM-based adipogenic induction media. Hence, we have demonstrated the isolated adipose-derived cell line can differentiate into mature adipocytes in FBS-free adipogenic induction media.

### Example 14

Titration of Adipogenic Induction Duration of pH9F-1x

To obtain high percentage of matured adipocytes and high level of lipids accumulation in a cost-effective manner, Ph9F-1x and Aj1C-1x were stained with AdipoRed Reagent at different timepoint of adipogenic induction with Essential 6 media, adipogenic induction cocktail, and LAOA. Staining result revealed that there is no significant difference in the percentage of matured adipocytes from Day 3 to Day 9 of adipogenic induction. Interestingly, the total lipid content in the matured adipocytes on Day 6 and Day 9 were significantly higher as compared to Day 0, Day 3, Day 4, and Day 5. Further, there is no significant difference in the total lipid content in the matured adipocytes on Day 6 and Day 9.

### Example 15

### Component Analysis of Adipogenic Induction Cocktail in Adipogenesis

Essential 6 basal was supplemented with LAOA and adipogenic induction cocktail to induce adipogenesis in the isolated adipose-derived cell lines. The adipogenic induction cocktail consists of insulin, which is already present in Essential 6, IBMX, dexamethasone, and indomethacin. To investigate the efficiency of each component in the adipogenic induction cocktail, Ph9F-1x was treated with Essential 6 media, fish serum, LAOA, and different combinations of dexamethasone, IBMX, and indomethacin. Staining result revealed that there is no significant difference in the percentage of matured adipocytes after treatment with different combinations of IBMX, dexamethasone, and indomethacin. It also revealed that treatment with IBMX and dexamethasone resulted in significantly higher total lipid content in the matured adipocytes as compared to treatment with IBMX, dexamethasone, and indomethacin.

### Example 16

### Identification of the Effective Concentration of LAOA During Adipogenesis

Although LAOA is one of the most important components during adipogenic induction of adipose-derived cell line, it is the most expensive component of our adipogenic induction media. To develop a cost-effective and efficient adipogenic induction media, Ph9F-1x was treated different concentration of LAOA. AdipoRed staining revealed that 50 μM and 100 μM of LAOA significantly enhanced the percentage of matured adipocytes and their total lipid content as compared to the absence of LAOA. In contrast, higher concentration (150 μM and 200 μM) of LAOA did not enhance the percentage of matured adipocytes and their total lipid content. 100 μM of LAOA appears to enhance the percentage of matured adipocytes and their total lipid content as compared to 50 μM of LAOA.

### Example 17

### Identification of the Timing of LAOA Supplementation During Adipogenic Induction

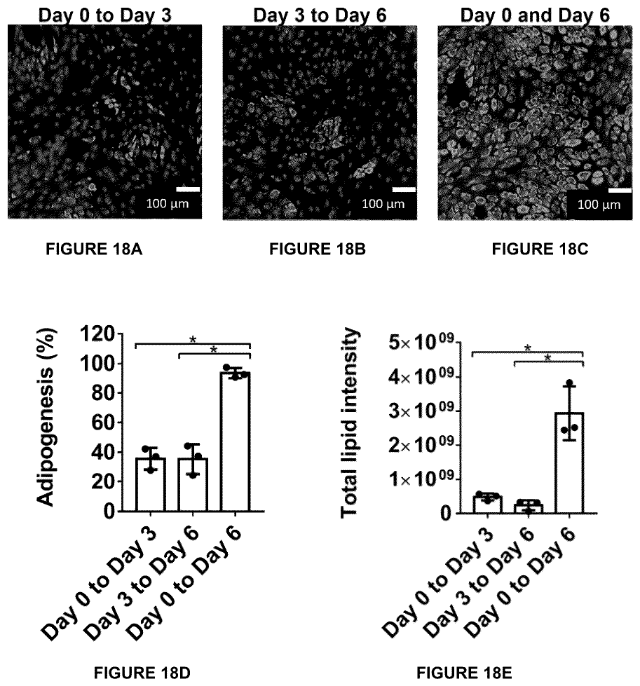

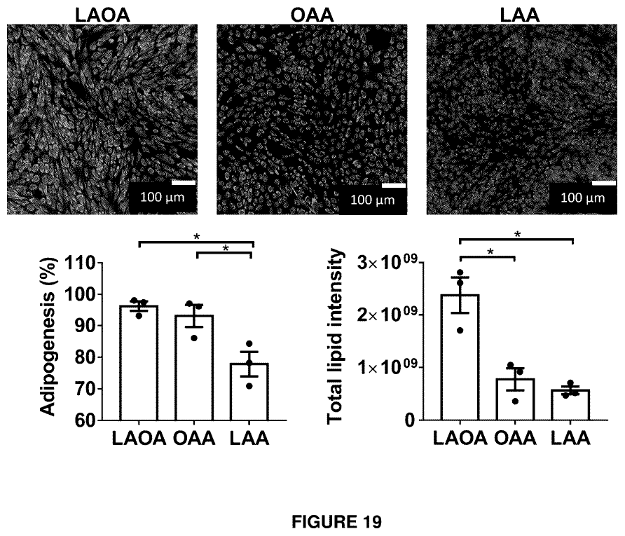

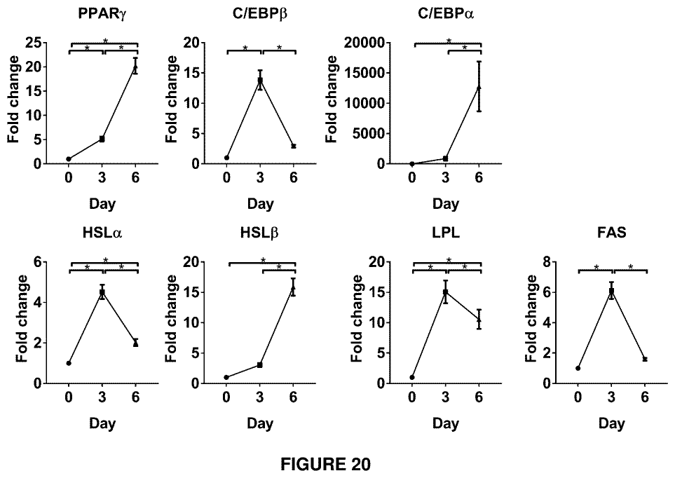

Thus far, to induce adipogenesis, isolated adipose-derived cell line was treated with Essential 6, fish serum, IBMX, dexamethasone, and LAOA for 3 days, followed by Essential 6, fish serum, and LAOA for another 3 days. To reduce the cost of producing cell-based fats, Ph9F-1x was treated with LAOA from Day 0 to Day 3 (FIG. 18A), from Day 3 to Day 6 (FIG. 18B), or Day 0 to Day 6 (FIG. 18C) of adipogenesis. AdipoRed staining revealed that supplementation of LAOA from Day 0 to Day 6 significantly enhanced its percentage of matured adipocytes (FIG. 17D) and their total lipid content (FIG. 18E) at the end of Day 6, as compared to when LAOA was supplemented from Day 0 to Day 3, or from Day 3 to Day 6 only. Hence, LAOA must be supplemented throughout adipogenic induction of fish adipose-derived cell line to obtain high percentage of matured adipocytes with the high level of lipid accumulation.

### Example 18

### Component Analysis of LAOA During Adipogenesis

LAOA is a mixture of linoleic acid (LAA)- and oleic acid (OAA)-conjugated Bovine Serum Albumin (BSA). The effectiveness of LAOA, OAA, and LAA in enhancing adipogenesis of fish adipose-derived cell lines were compared, respectively. AdipoRed staining revealed that LAOA and OAA significantly enhanced the percentage of matured adipocytes as compared to LAA. Furthermore, LAOA significantly enhanced the total lipid content of adipocytes as compared to OAA and LAA.

### Example 19

### Transcriptomic Analysis During Adipogenesis in Fish Adipose-Derived Cell Line

Apart from AdipoRed staining, the expression of various genes involved in adipogenesis and lipogenesis was also examined to reinforce that isolated adipose-derived cell line differentiates into matured adipocytes upon adipogenic induction. PPARγ is known as the master regulator of adipogenesis and the expression of PPARγ was shown to be significantly enhanced over different days of adipogenesis. Early adipogenic transcription factors, C/EBPβ and C/EBPδ, are known to be expressed in the early stages of adipogenesis and diminished in the late stages of adipogenesis. The expression of PPARγ, C/EBPβ, and C/EBPδ promotes the expression of C/EBPα in the later stages of adipogenesis. The expression of C/EBPβ was shown to be significantly enhanced on Day 3 and diminished on Day 6 of adipogenesis. Conversely, the expression of C/EBPδ was below detection limit. The expression of C/EBPα was also shown to be significantly enhanced on Day 6 of adipogenesis.

Further, the expression of various lipogenesis genes during adipogenesis of adipose-derived cell line was also investigated. The expression of HSLα and LPL were shown to be significantly enhanced in Day 3 and Day 6 of adipogenesis. The expression of HSLβ was significantly enhanced in Day 6 of adipogenesis and the expression of FAS was significantly enhanced on Day 3 of adipogenesis.

Taken together, the isolated adipose-derived cell line was shown to undergo adipogenesis upon adipogenic induction, as shown by the accumulation of intracellular lipid, expression of adipogenic transcription factors, and expression of lipogenic genes.

### Example 20

### Removal of Serum During Adipogenesis

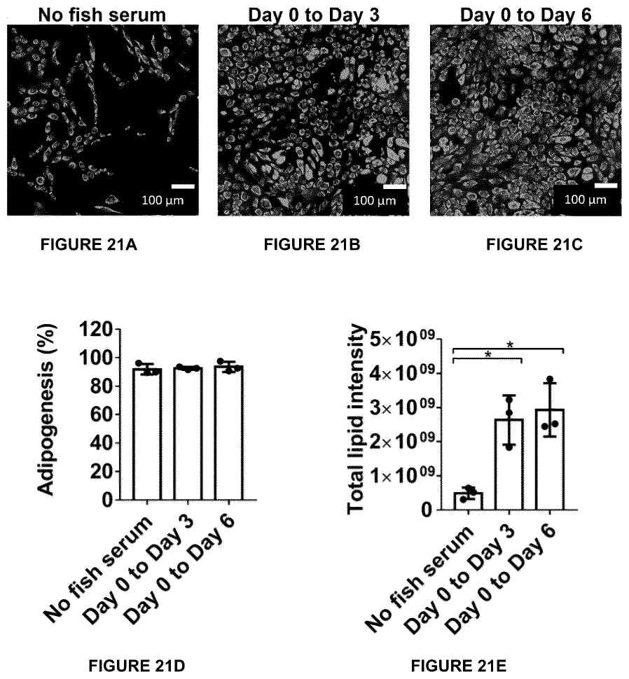

Serum is an important component of our growth media as the absence of fish serum significantly reduced cell proliferation of fish adipose-derived cell lines (FIG. 5). As the usage of serum is not sustainable and can potentially limit large scale production of cell-based fat, we investigated if the removal of fish serum on Day 0 or Day 3 would affect adipogenesis level of fish adipose-derived cell line. Staining results showed that the removal of fish serum on Day 0 resulted in markedly lower cell count (FIG. 21A) as compared to when fish serum was present from Day 0 to Day 3 (FIG. 21B), and Day 0 to Day 6 (FIG. 21C). Although AdipoRed staining showed no difference in the percentage of matured adipocytes regardless of the presence fish serum (FIG. 21D), the absence of fish serum during adipogenesis (removal on Day 0) resulted in significantly lower total lipid content as compared to when fish serum was present (FIG. 21E). Interestingly, the removal of fish serum on Day 3 does not give a significant difference in the percentage of matured adipocytes and total lipid content as compared to when fish serum was present throughout adipogenesis (Day 0 to 6). Hence, the data suggest that serum can be withdrawn during the last 3 days of adipogenesis without affecting the production of cell-based fats.

### Example 21

### Supplementation of Vitamins During Adipogenesis

Vitamin C and Vitamin D have been reported to regulate adipogenesis. Vitamin C is already present in some of the cell culture media, for example, Essential 6 media. Thus, the role of other vitamins in adipogenesis was also investigated. AdipoRed staining result revealed that 100 μM of α-tocopherol acetate, D-pantothenic acid, vitamin D3, or biotin did not significantly enhance the percentage of matured adipocytes and their total lipid content after 6 days. Interestingly, Vitamin D3 (intensity of 3.5×10⁹) enhanced the total lipid content of matured adipocyte (intensity of 1.9×10⁹) with borderline statistical significance.

### Example 22

Identification of the Duration of Adipogenesis of an Anguilla japonica Cell Line

After optimizing the adipogenesis conditions of Pangasianodon hypophthalmus cell line, we also determine the optimal adipogenesis conditions for Anguilla japonica cell line, Aj1C-1x. First, we investigated the suitable duration of adipogenic induction by comparing the level of lipid accumulation in Aj1C-1x during adipogenesis for 5 days. The data revealed that the fluorescence intensity of AdipoRed was significantly higher on Day 3 as compared to other days (FIG. 23). Thus, 3 days would be an effective duration of adipogenic induction to obtain the highest level of lipid intensity in matured Aj1C-1x adipocytes.

### Example 23

Identification of Effective Concentrations of LAOA Duration of Adipogenesis of Anguilla japonica Cell Line

As demonstrated previously in other Examples, LAOA is one of the key determinants of adipogenesis in Pangasianodon hypophthalmus cell lines. In another example, Aj1C-1x cell line were also treated with different concentrations of LAOA. AdipoRed staining revealed that 100 μM of LAOA significantly enhanced the percentage of matured adipocytes and their total lipid content as compared to 0 and 50 μM of LAOA (FIG. 24). Further, there is no significant difference in the percentage of matured adipocytes and their total lipid content between 100, 150, 200, and 500 μM of LAOA.

### Example 24

### Supplementation of Omega-3 Fatty Acid During Adipogenesis

Omega-3 fatty acids, such as docosahexaenoic acid (DHA) and eicosapentaenoic acid (EPA), play an important role in nutrition and cultivated meat as they are known to reduce the risks of cardiovascular diseases. DHA and EPA have been reported to affect different stages of adipogenesis and browning. Thus, the role of DHA and EPA during adipogenesis of Ph9F-1x was investigated by adding different concentrations of DHA and EPA during the final 3 days of adipogenesis. AdipoRed staining results showed that DHA (FIG. 25A) or EPA (FIG. 25B) did not significantly affect the percentage of matured adipocytes and their total lipid content. Staining results also revealed that 100 μM of DHA significantly reduced the nuclei count after adipogenesis (FIG. 25A).

To investigate the combinatory effects of DHA and EPA during adipogenesis, 50 μM of DHA and 50 μM of EPA were added during the final 3 days of adipogenesis. AdipoRed staining results revealed that the combination of 50 μM of DHA and 50 μM of EPA did not significantly affect the percentage of matured adipocytes and their total lipid content (FIG. 25C). Further, it did not affect the total nuclei count. Thus, 50 μM of DHA and 50 μM of EPA could be added to the adipogenic induction cocktail during the final 3 days of adipogenesis to enhance the level of omega-3 fatty acids in Ph9F-1x matured adipocytes.

### Example 25

### Transcriptomic Analysis During Adipogenesis in Presence of DHA and EPA

Gene expression of the master regulator of adipogenesis, PPARγ, and early adipogenic transcription factors, C/EBPβ were also investigated after differentiation of Ph9F-1x using adipogenic induction cocktail. Gene expression analysis revealed that the expression of both PPARγ and C/EBPβ were significantly enhanced on Day 3 and Day 6 of adipogenesis.

Taken together, Ph9F-1x were shown to differentiate into matured adipocytes using adipogenic induction cocktail as shown by the accumulation of intracellular lipid and expression of adipogenic genes.

### Example 26

Adipogenesis Potential of pH9F-1x at High Passage Number

The isolated adipose-derived cell lines, for example, Ph9F-1x, can grow for more than 100 passages as described earlier. To ascertain the adipogenic potential of Ph9F-1x at high passage numbers, we compared the percentage of adipogenesis and lipid accumulation of Ph9F-1x at passage 29 and 104. AdipoRed staining revealed that Ph9F-1x at passage 104 significantly enhanced the percentage of adipogenesis and lipid accumulation after stimulation with adipogenic induction media as compared to without stimulation (FIG. 27). Further, there is no significant difference in the percentage of adipogenesis and lipid accumulation between stimulated Ph9F-1x at passage 29 and 104. The data indicate that our fish cell line, Ph9F-1x, retains its adipogenic potential even at high passage number.

### Example 27

Measurement of DHA and EPA in Matured pH9F-1x Adipocytes.

Omega-3 fatty acids such as DHA and EPA are essential for a heathy diet and important components of cultivated meat as they are widely known to reduce the risk of cardiovascular diseases. The concentrations of omega-3 fatty acids were measured in the Ph9F-1x matured adipocytes. Mass spectrometry data revealed that Ph9F-1x matured adipocytes contained 3.86 g of DHA and 0.158 g of EPA for every 20 g of protein. As compared to the data published by United States Department of Agriculture, the total concentrations of DHA and EPA in Ph9F-1x matured adipocytes were higher than other premium fishes such as wild-caught Atlantic salmon and blue fin tuna. Hence, the matured adipocytes disclosed herein have high nutritional values as sources for cultivated meat.

TABLE 1
Concentrations of DHA and EPA in 20
gram of Ph9F-1x matured adipocytes.

| | DHA (gram per 20 | EPA (gram per 20 |
|---|---|---|
| | gram of protein) | gram of protein) |
| Ph9F-1x matured adipocytes | 3.86 | 0.16 |
| Wild-caught Atlantic salmon | 1.13* | 0.32* |
| Wild-caught blue fin tuna | 0.76* | 0.24* |

*Data of wild-caught Atlantic salmon and blue fin tuna were obtained from United States Department of Agriculture website.

### Example 28

Growth and Adipogenic Differentiation of pH9F-1x on Cytodex 1 Microcarrier

Microcarriers are widely used to support large scale culture of anchorage-dependent cell lines as it can substantially increase the scalability of cell culture, while reducing the costs and physical footprint requirement. Here, we have selected Cytodex 1 for expansion and differentiation of our isolated cell lines as it does not contain any animal components and can be produced in a sustainable manner. Ph9F-1x was grown on Cytodex 1 in spinner flask for 3 days and treated with TrypLE Express for 30 minutes. Trypan blue staining revealed that the viability of the cells were not affected by the treatment as more than 80% of the cells remained viable after 30 minutes. The data also revealed that 18 minutes of treatment produced the highest cell yield of 5.16×10⁷ cells (FIG. 28A), which was 7.7 fold higher than the initial cell seeding density. Nuclei staining also showed that Ph9F-1x adhered onto Cytodex 1 (FIG. 28B). Taken together, it is shown that the isolated fish cell line can be used for large scale culture as it is able to adhere and grow on Cytodex 1 microcarrier in 3-dimensional spinner flask.

The isolated cell lines were further investigated for their adipogenesis potential on Cytodex 1 microcarrier. AdipoRed staining result shows that stimulated Ph9F-1x cells were stained by AdipoRed whereas unstimulated cells were not stained by AdipoRed. The data indicates that Ph9F-1x can differentiate on Cytodex 1 microcarrier in 3-dimensional spinner flask.

1. A method of isolating adipose-derived cell lines from an animal, comprising:
(a) obtaining an adipose tissue sample from an animal;
(b) collecting stromal vascular cells from the adipose tissue sample of (a);
(c) expanding the stromal vascular cells of (b) in the presence of a serum;
(d) conducting clonal selection of the expanded stromal vascular cells based on the growth of the cells; and
(e) isolating one or more adipose-derived cell lines based on the selection result in (d).

2. The method of claim 1, wherein the isolated cell lines are characterized by not entering senescence.

3. The method of claim 1, wherein the isolated adipose-derived cell lines are isolated under (e) if the expanded stromal vascular cells under (d) continue to grow for about 16 to about 30 passages.

4. The method of claim 1, wherein the stromal vascular cells are expanded in the presence of a serum and a Fetal Bovine Serum (FBS).

5. The method of claim 1, wherein the adipose tissue is visceral fat tissue, a subcutaneous fat tissue, an intramuscular fat tissue, or an intermuscular fat tissue.

6.-7. (canceled)

8. A method of obtaining adipocytes from an animal, comprising:
(a) isolating adipose-derived cells from a tissue sample of the animal;
(b) culturing the adipose-derived cells in the presence of an adipogenesis induction; and
(c) obtaining adipocytes from the cell culture of (b);
wherein the adiogenesis induction composition comprises: a linoleic acid-oleic acid albumin (LAGA), an insulin, a serum, and optionally at least one, at least two, or all of 3-isobutyl-1-methylxanthine (IBMX)s, dexamethasone, and indomethacin; optionally wherein the adipogenesis induction composition further comprises a basic fibroblast growth factor (bFGF), and a Fetal Bovine Serum (FBS).

9. The method of claim 8, wherein the adipose-derived cells are further cultured in the presence of vitamins, and omega-3 fatty acids.

10. The method of claim 8, wherein the adipose-derived cells of step (b) are cultured for at least about 3 days, or about 6 days, or about 9 days.

11. The method of claim 8, wherein the serum, 3-isobutyl-1-methylxanthine (IBMX), dexamethasone, and indomethacin are removed after about 3 days of culturing under (b).

12. The method of claim 8, wherein the method is carried out in a bioreactor.

13. The method of claim 12, wherein the bioreactor comprises a microcarrier.

14. The method of claim 8, wherein step (a) is carried out according to the method comprising:
(i) obtaining an adipose tissue sample from an animal;
(ii) collecting stromal vascular cells from the adipose tissue sample of (i);
(iii) expanding the stromal vascular cells of (ii) in the presence of a serum;
(iv) conducting clonal selection of the expanded stromal vascular cells based on the growth of the cells; and
(v) isolating one or more adipose-derived cell lines based on the selection result in (iv).

15.-18. (canceled)

19. The method of claim 1, wherein the animal is a poultry, a livestock, or a fish.

20. The method of claim 19, wherein the fish is a bony fish (teleostomi) or a cartilaginous fish (chondrichthyes).

21. The method of claim 19, wherein the fish is selected form the following group comprising: Pangasianodon hypophthalmus, Anguilla japonica, Scortum barcoo, and Lates calcarifer.

22. The method of claim 1, wherein the serum is a serum obtained from an animal of the same phylum.

23. The method of claim 1, wherein the serum is a serum obtained from an animal of the same species.

24. The method of claim 1, wherein the serum is a fish serum.

25.-30. (canceled)

31. The method of claim 8, wherein the serum is a serum obtained from an animal of the same phylum.

32. The method of claim 8, wherein the serum is a serum obtained from an animal of the same species.

33. The method of claim 8, wherein the serum is a fish serum.
